(19) United States
(12) Patent Application Publication
(10) Pub. No.: US 2024/0208935 A1
(43) Pub. Date: Jun. 27, 2024

(54) COMPOSITIONS AND METHODS FOR TREATING NEURODEGENERATIVE DISEASES

(71) Applicant: The Regents of the University of California, Oakland, CA (US)

(72) Inventors: Varghese John, Los Angeles, CA (US); Istvan Mody, Los Angeles, CA (US); Jesus J. Campagna, Playa Del Rey, CA (US); Barbara Jagodzinska, Redwood City, CA (US); Xiaofei Wei, Los Angeles, CA (US)

(21) Appl. No.: 18/287,586

(22) PCT Filed: Apr. 21, 2022
(86) PCT No.: PCT/US22/25732

§ 371 Date: Oct. 19, 2023

Related U.S. Application Data
(60) Provisional application No. 63/177,710, filed on Apr. 21, 2021.

(51) Int. Cl. C07D 401/14; A61K 31/4155; A61K 31/454; A61K 31/4709; A61K 31/4725; A61K 31/55; A61P 25/28; C07D 401/04; C07D 403/04; C07D 405/14

(52) U.S. Cl. CPC C07D 401/14; A61K 31/4155; A61K 31/454; A61K 31/4709; A61K 31/4725; A61K 31/55; A61P 25/28; C07D 401/04; C07D 403/04; C07D 405/14

(57) ABSTRACT

The present disclosure relates to compounds which increase γ-oscillations in the brain. The disclosure further relates to methods of treating neurodegenerative diseases or disorders with the compounds disclosed herein.

### RELATED APPLICATIONS

The instant application claims priority to U.S. Provisional Patent Application No.: 63/177,710, filed Apr. 21, 2021, the contents of which are hereby fully incorporated by reference herein.

### BACKGROUND

Alzheimer's disease (AD) has a high annual incidence rate that continues to increase. For instance, six million people are affected in the US, and over 24 million world-wide. Furthermore, AD incidence is expected to double every 20 years until 2047. This creates an enormous burden on society and AD is becoming a public health emergency. A great deal about the fundamental pathology of AD is known, yet there are no drugs to restore cognition and memory in AD patients. It is becoming evident that AD may result from positive feedback mechanisms exerted by both Aβ and the imminent changes in neuronal excitability that accompany the early stages of the disease when mild cognitive impairment (MCI) is present, to the extent it can be monitored in mouse models of AD.

The brain generates rhythms of various frequencies that result from the orchestrated activities of well-defined local neuronal circuits. Of these rhythms, γ-oscillations (30-120 Hz) are the most important for brain mechanisms inexorably linked to cognitive processes and working memory and a decline of these is present in numerous neurological and psychiatric disorders including MCI, AD, epilepsy, traumatic brain injury (TBI), depression, and schizophrenia. However, to date, there are no FDA approved pharmaceuticals which stimulate γ-oscillations.

In view of the foregoing, there is an ongoing need for new compounds to treat neurological disorders, such as AD, by simulating γ-oscillations within the brain.

### SUMMARY OF THE INVENTION

In one aspect, the present disclosure provides compounds of Formula I:

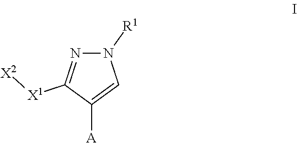

or a pharmaceutically acceptable salt thereof, wherein:
A is heterocyclyl;
X¹ is alkylene, carbonyl, N(R₂), or O;
X² is aryl or heteroaryl;
R¹ is alkyl, hydroxyl, or alkyloxy; and
R² is H, alkyl, or aralkyl.

In another aspect, the present disclosure provides methods of treating a neurodegenerative disease or disorder in a subject comprising administering a compound of the disclosure or a pharmaceutically acceptable salt thereof to the subject.

### BRIEF DESCRIPTION OF THE DRAWINGS

FIG. 1A shows a plot of the inhibited fraction (efficacy of inhibition) of the tonic current vs the same for the phasic current by DDL-920 in cortical PV+INs. The small dots are from individual cell recordings while the large dots are the average values with the SD bars extending in both directions. The dashed line has a unity slope and thus indicates no specificity of the compound for either tonic or phasic inhibition.

FIG. 1B shows effects of 1 nM DDL-920 on in vitro KA-induced γ-oscillations in the hippocampal CA3 pyramidal layer. The Morlet wavelets show the large enhancement of the magnitude of 40-50 Hz oscillations during 2 min epochs before and after the application of 1 nM DDL-920. The FFTs of the two epochs are shown on the lower panel with the areas under the curve indicated between 30-120 Hz. The ratio of the drug/control areas is 4.26.

FIGS. 2 & 3 show that a 10 min perfusion of 100 nM DDL-920 resulted in a significant increase in γ-oscillations in the CA3 region hippocampal slices.

FIG. 4 shows the DDL-920 concentrations achieved in the brains of mice at the indicated times after 3 different doses, 1, 5, 10 mg/kg (mkd), of SQ injections.

FIG. 5 shows a randomly chosen 1 s long epoch from the analyzed periods before and after DDL-920 injection. Exemplary θ- and γ-oscillation periods are indicated by braces. Larger amplitudes of the γ-oscillations followed the injection.

FIG. 6 shows the modulation index (MI), γ-oscillation frequency, θ-oscillation frequency, and γ-oscillation RMS (power), respectively, plotted as the values after the injections, vs the values before the injections (DDL-920: grey n=4 mice, 5 injections; SAL: black, n=3 mice).

### DETAILED DESCRIPTION OF THE INVENTION

Brain stimulation at frequencies resembling γ-oscillations using sensory inputs or transcranial modalities reduce the plaque burden in mouse models of advanced Alzheimer's disease. However, such stimuli are ineffective in MCI as they cannot reach all brain areas, and their duration and timing are artificial. In contrast, the approached disclosed herein will restore cognitive performance by engaging the brain's naturally occurring γ-oscillations by improving the function of PV+INs. These neurons are critically implicated in the generation of most rhythmic neuronal activities related to cognitive tasks in the brain, including γ-oscillations and the tight phase amplitude coupling (PAC) between the phase of θ-oscillations (5-12 Hz) and γ-amplitude. In both humans and experimental animals such oscillatory events are considered to be key mechanisms for cognition and short-term/working memory.

In certain aspects, the present disclosure provides compounds of Formula I:

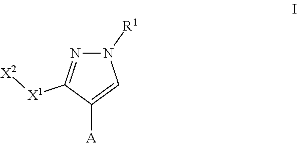

or a pharmaceutically acceptable salt thereof, wherein:
A is heterocyclyl;
X¹ is alkylene, carbonyl, N(R²), or O;
X² is aryl or heteroaryl;
R¹ is alkyl, hydroxyl, or alkoxy; and
R² is H, alkyl, or aralkyl.

In certain embodiments, the compound is not

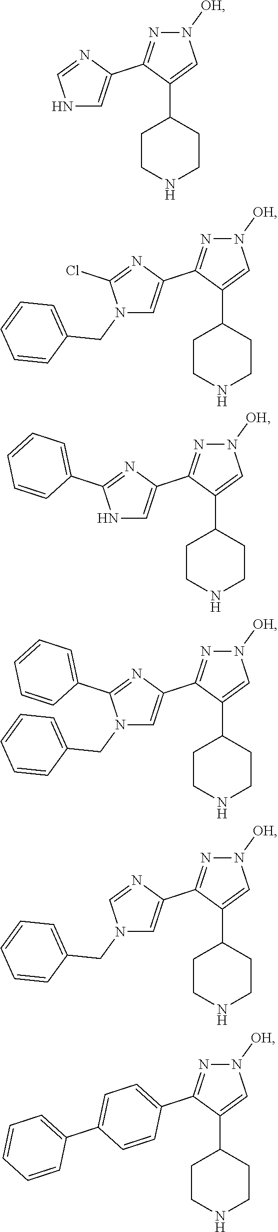

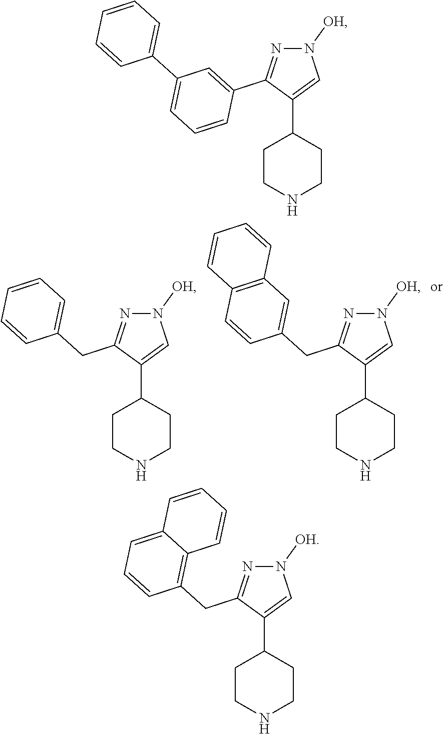

In certain embodiments, the compound is not a salt of

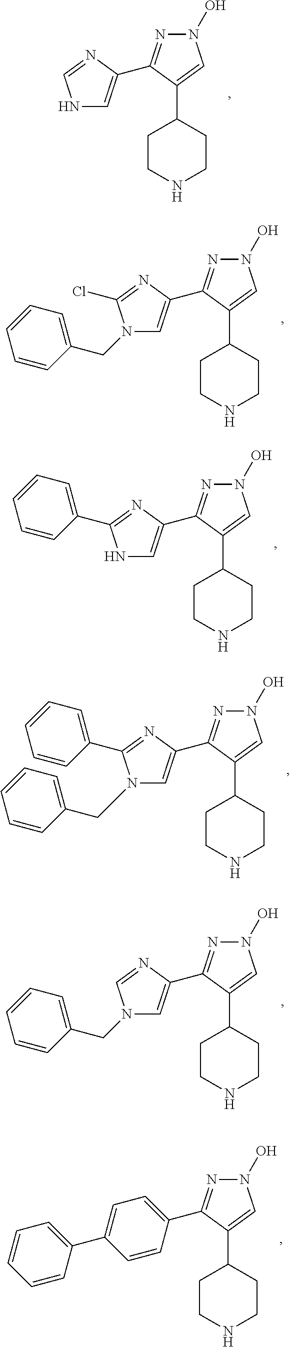 [SMILES: On1cc(C2CCNCC2)c(-c2c[nH]cn2)n1 ; On1cc(C2CCNCC2)c(-c2cn(Cc3ccccc3)c(Cl)n2)n1 ; On1cc(C2CCNCC2)c(-c2c[nH]c(-c3ccccc3)n2)n1 ; On1cc(C2CCNCC2)c(-c2cn(Cc3ccccc3)c(-c3ccccc3)n2)n1 ; On1cc(C2CCNCC2)c(-c2cn(Cc3ccccc3)cn2)n1 ; On1cc(C2CCNCC2)c(-c2ccc(-c3ccccc3)cc2)n1]

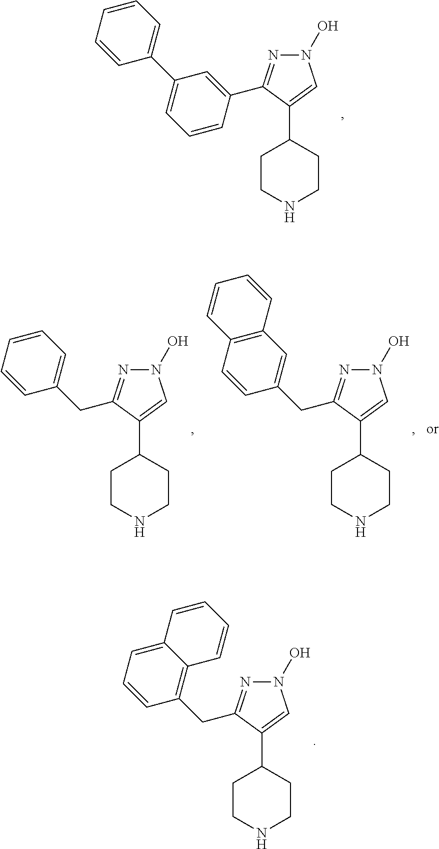

In certain embodiments, A is a 4-8 membered nitrogen containing heterocyclyl. In certain embodiments, A is azetedinyl, pyrrolidinyl, or piperidinyl. In certain preferred embodiments, A is N-methylpiperidinyl, N-ethylpiperidinyl, or N-propylpiperidinyl.

In certain preferred embodiments, R¹ is hydroxyl.

In certain embodiments, X¹ is alkylene. In certain preferred embodiments, X¹ is methylenyl. In other preferred embodiments, X¹ is carbonyl. In yet other preferred embodiments, X¹ is N(R²).

In certain embodiments, X¹ is substituted with alkyl, alkenyl, alkynyl, halo, hydroxyl, carboxyl, acyl, acetyl, ester, thioester, alkoxy, phosphoryl, amino, amide, cyano, nitro, azido, alkylthio, cycloalkyl, alkylsulfonyl, and sulfonamide. In certain preferred embodiments, X¹ is substituted with alkyl.

In certain embodiments, R² is H.

In certain embodiments, the compound has a structure represented by formula Ia:

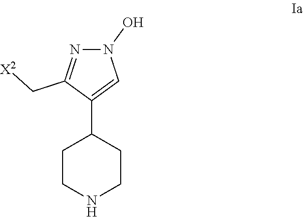

or a pharmaceutically acceptable salt thereof.

In certain embodiments, X² is aryl. In certain preferred embodiments, X² is phenyl, naphthyl, dihydrobenzodioxinyl, or benzodioxolyl. In other embodiments, X² is heteroaryl. In certain preferred embodiments, X² is quinolinyl or isoquinolinonyl.

In certain embodiments, X² is substituted with alkyl, alkenyl, alkynyl, halo, hydroxyl, thiol, carboxyl, acyl, acetyl, ester, thioester, alkoxy, phosphoryl, amino, amide, cyano, nitro, azido, alkylthio, cycloalkyl, alkylsulfonyl, and sulfonamide. In certain embodiments, X² is substituted with alkyl (e.g., methyl, ethyl, propyl, isopropyl, or butyl), halo (e.g., fluoro or chloro), alkenyl (e.g., allyl), alkyloxy (e.g., methoxy), alkylthio (e.g., methylthio), thiol, or aryl (e.g., phenyl, such as methyl or fluorophenyl).

In certain preferred embodiments, the substituent on X² is in the para position as compared to X¹.

In certain embodiments, the compound is selected from

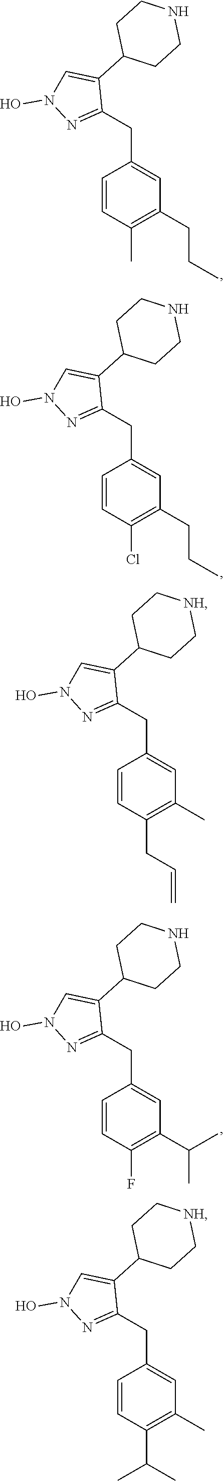

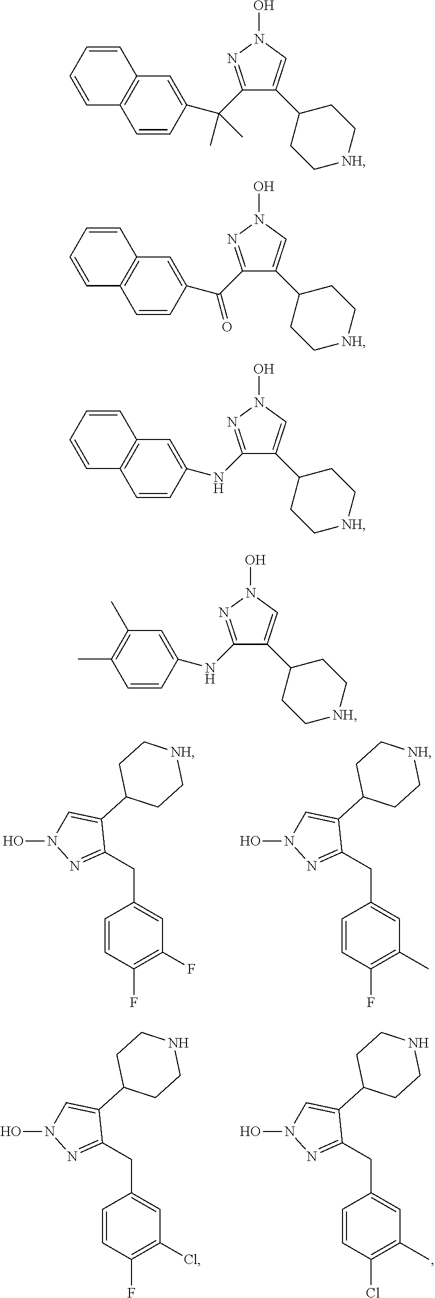

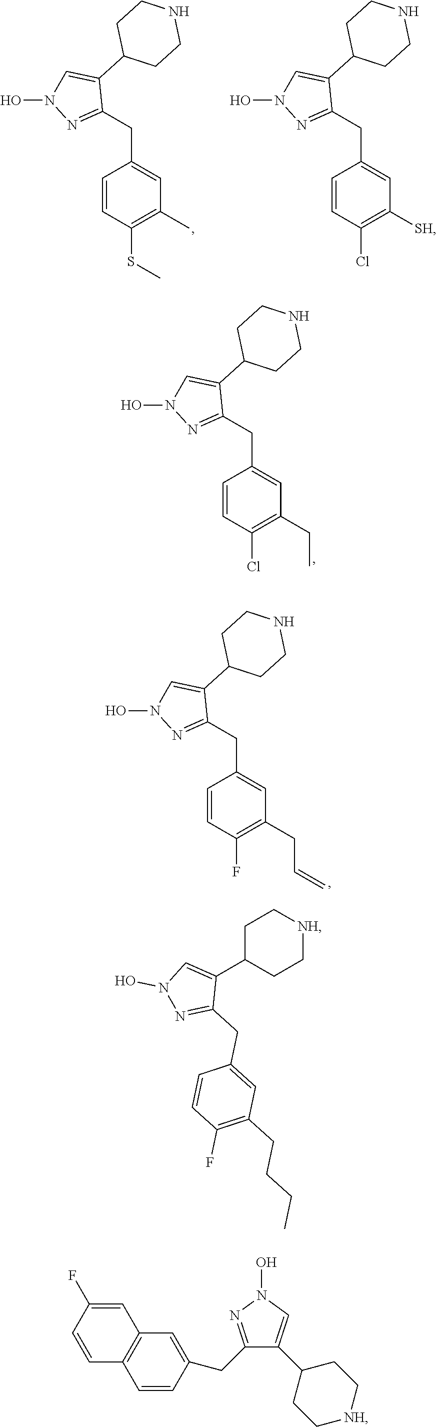

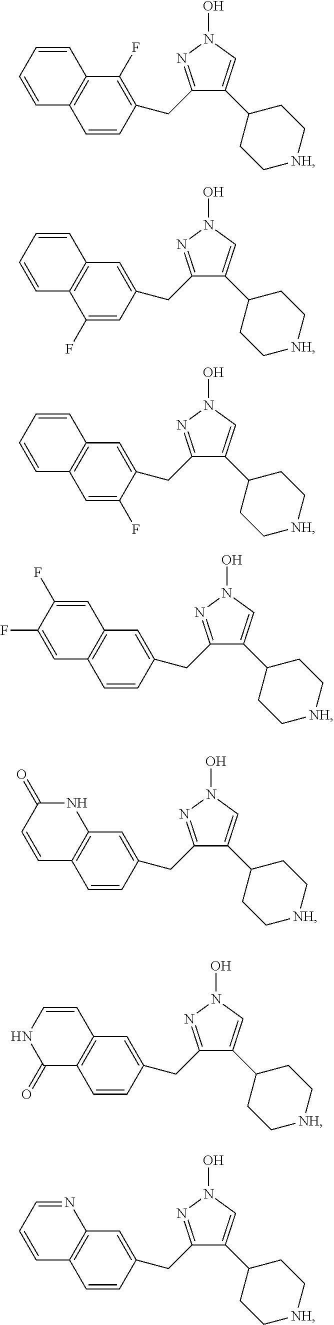

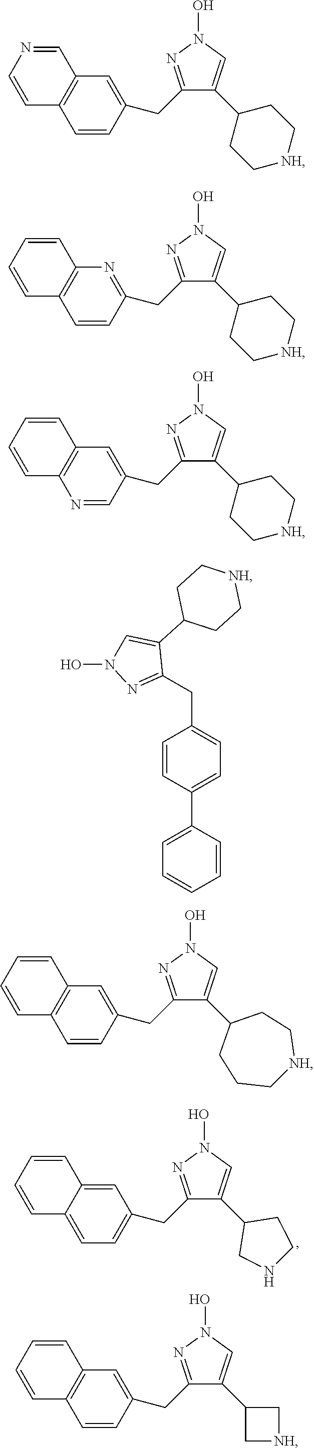

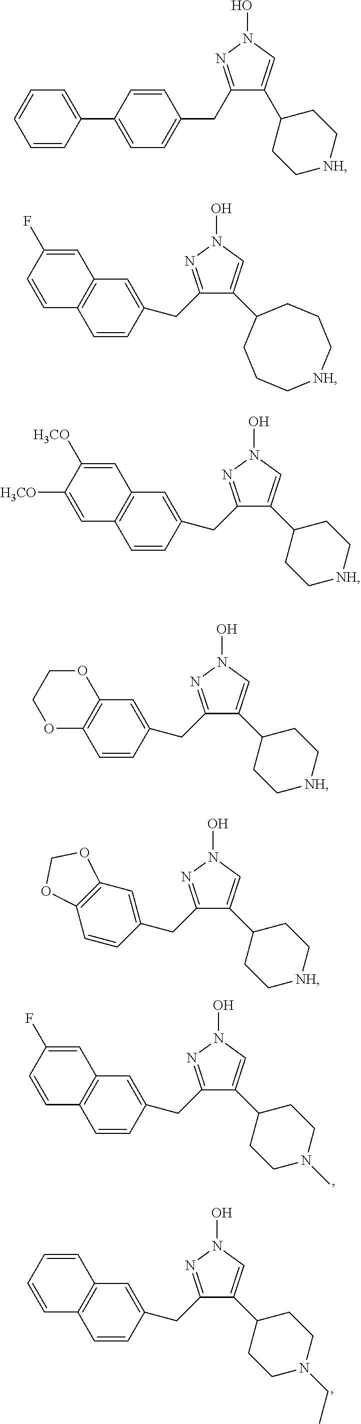

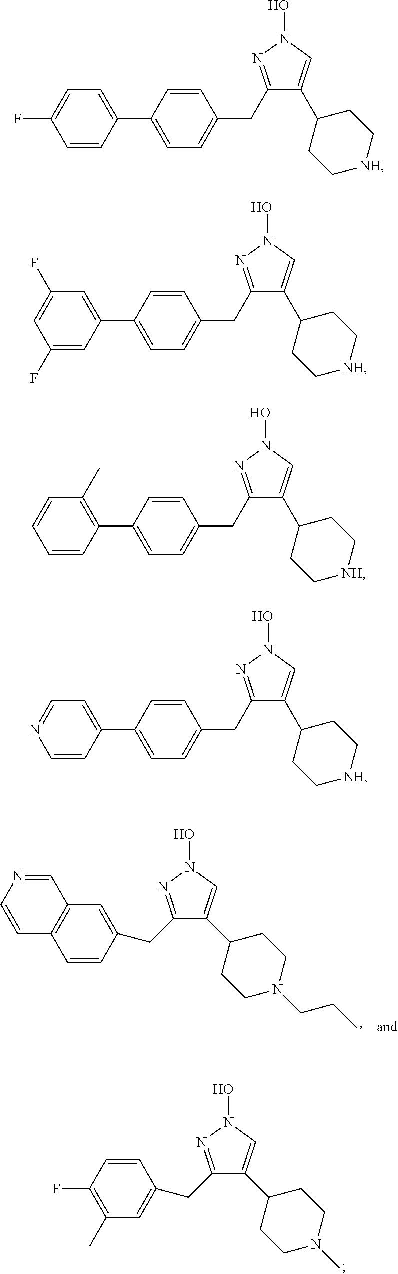

or a pharmaceutically acceptable salt thereof.

In another aspect, the present disclosure provides pharmaceutical compositions comprising a compound of the disclosure or a pharmaceutically acceptable salt thereof and a pharmaceutically acceptable excipient.

In another aspect, the present disclosure provides methods of treating a neurodegenerative disease or disorder in a subject comprising administering a compound of the disclosure or a pharmaceutically acceptable salt thereof to the subject.

In another aspect, the present disclosure provides methods of treating a neurodegenerative disease or disorder in a subject, comprising administering a compound or a pharmaceutically acceptable salt thereof to the subject, wherein the compound is selected from

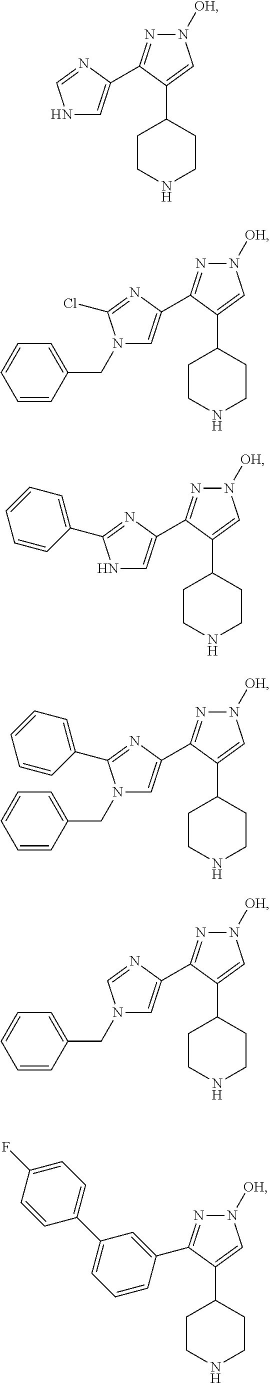 [SMILES: On1cc(C2CCNCC2)c(-c2c[nH]cn2)n1 ; On1cc(C2CCNCC2)c(-c2cn(Cc3ccccc3)c(Cl)n2)n1 ; On1cc(C2CCNCC2)c(-c2c[nH]c(-c3ccccc3)n2)n1 ; On1cc(C2CCNCC2)c(-c2cn(Cc3ccccc3)c(-c3ccccc3)n2)n1 ; On1cc(C2CCNCC2)c(-c2cn(Cc3ccccc3)cn2)n1 ; On1cc(C2CCNCC2)c(-c2cccc(-c3ccc(F)cc3)c2)n1]

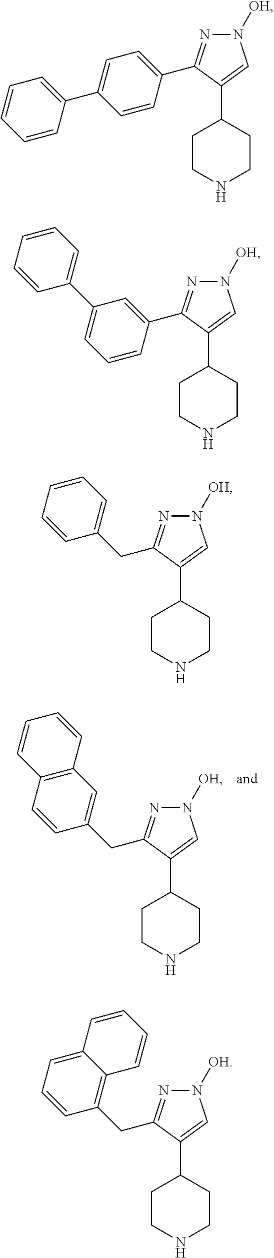 [SMILES: On1cc(C2CCNCC2)c(-c2ccc(-c3ccccc3)cc2)n1 ; On1cc(C2CCNCC2)c(-c2cccc(-c3ccccc3)c2)n1 ; On1cc(C2CCNCC2)c(Cc2ccccc2)n1 ; On1cc(C2CCNCC2)c(Cc2ccc3ccccc3c2)n1 ; "and" ; On1cc(C2CCNCC2)c(Cc2cccc3ccccc23)n1]

In certain embodiments, the neurodegenerative disease or disorder is Alzheimer's disease, Parkinson's disease, multiple sclerosis, stroke, amyotrophic lateral sclerosis, cerebellar ataxia, frontotemporal dementia, prion disease, Huntington's Disease, cerebral ischaemia, idiopathic Morbus Parkinson, Parkinson syndrome, Morbus Alzheimers, cerebral dementia syndrome, infection-induced neurodegeneration disorders, AIDS-encephalopathy, Creutzfeld-Jakob disease, encephalopathies induced by rubiola and herpes viruses and borrelioses, metabolic-toxic neurodegenerative disorders, hepatic-, alcoholic-, hypoxic-, hypo- or hyperglycemically-induced encephalopathies, encephalopathies induced by solvents or pharmaceuticals, degenerative retina disorders, trauma-induced brain damage, cerebral hyperexcitability symptoms, cerebral hyperexcitability states, neurodegenerative syndromes of the peripheral nervous system, peripheral nerve injury, or spinal cord injury. In certain embodiments, the neurodegenerative disease or disorder is Autism Spectrum Disorder (ASD), Rett syndrome, intellectual disability arising from Fragile X syndrome, intellectual disability arising from variants of Fragile X syndrome, schizophrenia, depression, major depressive disorder, or post-traumatic stress disorder (PTSD). In certain embodiments, the neurodegenerative disease or disorder is Alzheimer's disease, Parkinson's disease, Huntington's disease, Lewy body dementia, frontotemporal dementia, amyotrophic lateral sclerosis, multiple sclerosis, progressive supranuclear palsy, or age related cognitive decline. In certain embodiments, the neurodegenerative disease or disorder is age related mild cognitive impairment (MCI). In certain preferred embodiments, the neurodegenerative disease or disorder is Alzheimer's disease.

In another aspect, the present disclosure provides methods of increasing γ-oscillations in a subject comprising administering a compound of the disclosure or a pharmaceutically acceptable salt thereof to the subject.

In another aspect, the present disclosure provides methods of increasing γ-oscillations in a subject comprising administering a compound or a pharmaceutically acceptable salt thereof to the subject, wherein the compound is selected from

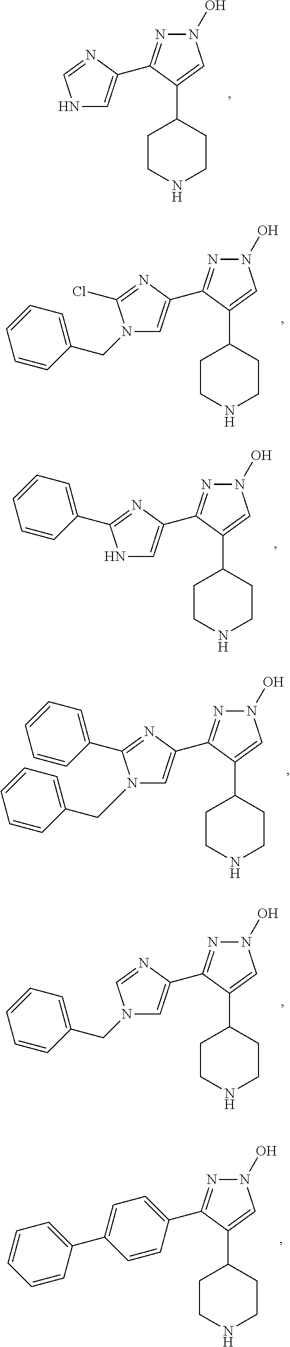 [SMILES: On1cc(C2CCNCC2)c(-c2c[nH]cn2)n1 ; On1cc(C2CCNCC2)c(-c2cn(Cc3ccccc3)c(Cl)n2)n1 ; On1cc(C2CCNCC2)c(-c2c[nH]c(-c3ccccc3)n2)n1 ; On1cc(C2CCNCC2)c(-c2cn(Cc3ccccc3)c(-c3ccccc3)n2)n1 ; On1cc(C2CCNCC2)c(-c2cn(Cc3ccccc3)cn2)n1 ; On1cc(C2CCNCC2)c(-c2ccc(-c3ccccc3)cc2)n1]

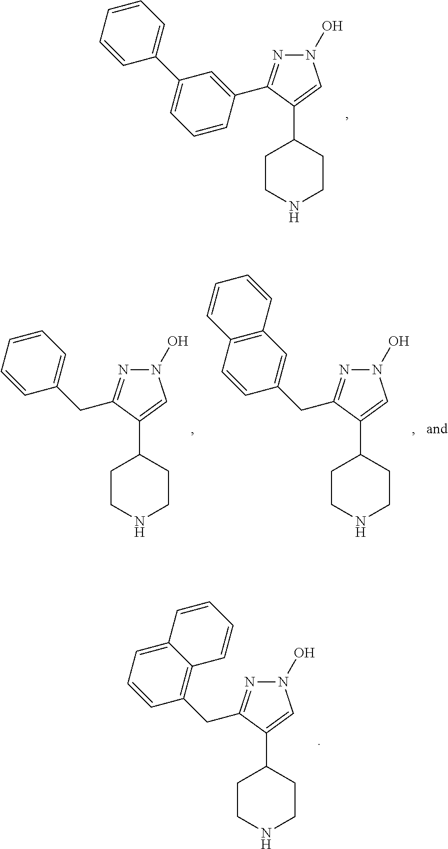

### Pharmaceutical Compositions

The compositions and methods of the present invention may be utilized to treat an individual in need thereof. In certain embodiments, the individual is a mammal such as a human, or a non-human mammal. When administered to an animal, such as a human, the composition or the compound is preferably administered as a pharmaceutical composition comprising, for example, a compound of the invention and a pharmaceutically acceptable carrier. Pharmaceutically acceptable carriers are well known in the art and include, for example, aqucous solutions such as water or physiologically buffered saline or other solvents or vehicles such as glycols, glycerol, oils such as olive oil, or injectable organic esters. In preferred embodiments, when such pharmaceutical compositions are for human administration, particularly for invasive routes of administration (i.e., routes, such as injection or implantation, that circumvent transport or diffusion through an epithelial barrier), the aqueous solution is pyrogen-free, or substantially pyrogen-free. The excipients can be chosen, for example, to effect delayed release of an agent or to selectively target one or more cells, tissues or organs. The pharmaceutical composition can be in dosage unit form such as tablet, capsule (including sprinkle capsule and gelatin capsule), granule, lyophile for reconstitution, powder, solution, syrup, suppository, injection or the like. The composition can also be present in a transdermal delivery system, e.g., a skin patch. The composition can also be present in a solution suitable for topical administration, such as a lotion, cream, or ointment.

A pharmaceutically acceptable carrier can contain physiologically acceptable agents that act, for example, to stabilize, increase solubility or to increase the absorption of a compound such as a compound of the invention. Such physiologically acceptable agents include, for example, carbohydrates, such as glucose, sucrose or dextrans, antioxidants, such as ascorbic acid or glutathione, chelating agents, low molecular weight proteins or other stabilizers or excipients. The choice of a pharmaceutically acceptable carrier, including a physiologically acceptable agent, depends, for example, on the route of administration of the composition. The preparation or pharmaceutical composition can be a selfemulsifying drug delivery system or a selfmicroemulsifying drug delivery system. The pharmaceutical composition (preparation) also can be a liposome or other polymer matrix, which can have incorporated therein, for example, a compound of the invention. Liposomes, for example, which comprise phospholipids or other lipids, are nontoxic, physiologically acceptable and metabolizable carriers that are relatively simple to make and administer.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically acceptable carrier” as used herein means a pharmaceutically acceptable material, composition or vehicle, such as a liquid or solid filler, diluent, excipient, solvent or encapsulating material. Each carrier must be “acceptable” in the sense of being compatible with the other ingredients of the formulation and not injurious to the patient. Some examples of materials which can serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

A pharmaceutical composition (preparation) can be administered to a subject by any of a number of routes of administration including, for example, orally (for example, drenches as in aqueous or non-aqueous solutions or suspensions, tablets, capsules (including sprinkle capsules and gelatin capsules), boluses, powders, granules, pastes for application to the tongue); absorption through the oral mucosa (e.g., sublingually); subcutaneously; transdermally (for example as a patch applied to the skin); and topically (for example, as a cream, ointment or spray applied to the skin). The compound may also be formulated for inhalation. In certain embodiments, a compound may be simply dissolved or suspended in sterile water. Details of appropriate routes of administration and compositions suitable for same can be found in, for example, U.S. Pat. Nos. 6,110,973, 5,763,493, 5,731,000, 5,541,231, 5,427,798, 5,358,970 and 4,172,896, as well as in patents cited therein.

The formulations may conveniently be presented in unit dosage form and may be prepared by any methods well known in the art of pharmacy. The amount of active ingredient which can be combined with a carrier material to produce a single dosage form will vary depending upon the host being treated, the particular mode of administration. The amount of active ingredient that can be combined with a carrier material to produce a single dosage form will generally be that amount of the compound which produces a therapeutic effect. Generally, out of one hundred percent, this amount will range from about 1 percent to about ninety-nine percent of active ingredient, preferably from about 5 percent to about 70 percent, most preferably from about 10 percent to about 30 percent.

Methods of preparing these formulations or compositions include the step of bringing into association an active compound, such as a compound of the invention, with the carrier and, optionally, one or more accessory ingredients. In general, the formulations are prepared by uniformly and intimately bringing into association a compound of the present invention with liquid carriers, or finely divided solid carriers, or both, and then, if necessary, shaping the product.

Formulations of the invention suitable for oral administration may be in the form of capsules (including sprinkle capsules and gelatin capsules), cachets, pills, tablets, lozenges (using a flavored basis, usually sucrose and acacia or tragacanth), lyophile, powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, cach containing a predetermined amount of a compound of the present invention as an active ingredient. Compositions or compounds may also be administered as a bolus, electuary or paste.

To prepare solid dosage forms for oral administration (capsules (including sprinkle capsules and gelatin capsules), tablets, pills, dragees, powders, granules and the like), the active ingredient is mixed with one or more pharmaceutically acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, cetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof; (10) complexing agents, such as, modified and unmodified cyclodextrins; and (11) coloring agents. In the case of capsules (including sprinkle capsules and gelatin capsules), tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered compound moistened with an inert liquid diluent.

The tablets, and other solid dosage forms of the pharmaceutical compositions, such as dragees, capsules (including sprinkle capsules and gelatin capsules), pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be sterilized by, for example, filtration through a bacteria-retaining filter, or by incorporating sterilizing agents in the form of sterile solid compositions that can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions that can be used include polymeric substances and waxes. The active ingredient can also be in micro-encapsulated form, if appropriate, with one or more of the above-described excipients.

Liquid dosage forms useful for oral administration include pharmaceutically acceptable emulsions, lyophiles for reconstitution, microemulsions, solutions, suspensions, syrups and elixirs. In addition to the active ingredient, the liquid dosage forms may contain inert diluents commonly used in the art, such as, for example, water or other solvents, cyclodextrins and derivatives thereof, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, coloring, perfuming and preservative agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Dosage forms for the topical or transdermal administration include powders, sprays, ointments, pastes, creams, lotions, gels, solutions, patches and inhalants. The active compound may be mixed under sterile conditions with a pharmaceutically acceptable carrier, and with any preservatives, buffers, or propellants that may be required.

The ointments, pastes, creams and gels may contain, in addition to an active compound, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to an active compound, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

Transdermal patches have the added advantage of providing controlled delivery of a compound of the present invention to the body. Such dosage forms can be made by dissolving or dispersing the active compound in the proper medium. Absorption enhancers can also be used to increase the flux of the compound across the skin. The rate of such flux can be controlled by either providing a rate controlling membrane or dispersing the compound in a polymer matrix or gel.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrasternal injection and infusion. Pharmaceutical compositions suitable for parenteral administration comprise one or more active compounds in combination with one or more pharmaceutically acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

Examples of suitable aqueous and nonaqueous carriers that may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents that delay absorption such as aluminum monostearate and gelatin.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution, which, in turn, may depend upon crystal size and crystalline form. Alternatively, delayed absorption of a parenterally administered drug form is accomplished by dissolving or suspending the drug in an oil vehicle.

Injectable depot forms are made by forming microencapsulated matrices of the subject compounds in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions that are compatible with body tissue.

For use in the methods of this invention, active compounds can be given per se or as a pharmaceutical composition containing, for example, 0.1 to 99.5% (more preferably, 0.5 to 90%) of active ingredient in combination with a pharmaceutically acceptable carrier.

Methods of introduction may also be provided by rechargeable or biodegradable devices. Various slow release polymeric devices have been developed and tested in vivo in recent years for the controlled delivery of drugs, including proteinaceous biopharmaceuticals. A variety of biocompatible polymers (including hydrogels), including both biodegradable and non-degradable polymers, can be used to form an implant for the sustained release of a compound at a particular target site.

Actual dosage levels of the active ingredients in the pharmaceutical compositions may be varied so as to obtain an amount of the active ingredient that is effective to achieve the desired therapeutic response for a particular patient, composition, and mode of administration, without being toxic to the patient.

The selected dosage level will depend upon a variety of factors including the activity of the particular compound or combination of compounds employed, or the ester, salt or amide thereof, the route of administration, the time of administration, the rate of excretion of the particular compound(s) being employed, the duration of the treatment, other drugs, compounds and/or materials used in combination with the particular compound(s) employed, the age, sex. weight, condition, general health and prior medical history of the patient being treated, and like factors well known in the medical arts.

A physician or veterinarian having ordinary skill in the art can readily determine and prescribe the therapeutically effective amount of the pharmaceutical composition required. For example, the physician or veterinarian could start doses of the pharmaceutical composition or compound at levels lower than that required in order to achieve the desired therapeutic effect and gradually increase the dosage until the desired effect is achieved. By “therapeutically effective amount” is meant the concentration of a compound that is sufficient to elicit the desired therapeutic effect. It is generally understood that the effective amount of the compound will vary according to the weight, sex, age, and medical history of the subject. Other factors which influence the effective amount may include, but are not limited to, the severity of the patient's condition, the disorder being treated, the stability of the compound, and, if desired, another type of therapeutic agent being administered with the compound of the invention. A larger total dose can be delivered by multiple administrations of the agent. Methods to determine efficacy and dosage are known to those skilled in the art (Isselbacher et al. (1996) Harrison's Principles of Internal Medicine 13 ed., 1814-1882, herein incorporated by reference).

In general, a suitable daily dose of an active compound used in the compositions and methods of the invention will be that amount of the compound that is the lowest dose effective to produce a therapeutic effect. Such an effective dose will generally depend upon the factors described above.

If desired, the effective daily dose of the active compound may be administered as one, two, three, four, five, six or more sub-doses administered separately at appropriate intervals throughout the day, optionally, in unit dosage forms. In certain embodiments of the present invention, the active compound may be administered two or three times daily. In preferred embodiments, the active compound will be administered once daily.

The patient receiving this treatment is any animal in need, including primates, in particular humans; and other mammals such as equines, cattle, swine, sheep, cats, and dogs; poultry; and pets in general.

In certain embodiments, compounds of the invention may be used alone or conjointly administered with another type of therapeutic agent.

The present disclosure includes the use of pharmaceutically acceptable salts of compounds of the invention in the compositions and methods of the present invention. In certain embodiments, contemplated salts of the invention include, but are not limited to, alkyl, dialkyl, trialkyl or tetra-alkyl ammonium salts. In certain embodiments, contemplated salts of the invention include, but are not limited to, L-arginine, benenthamine, benzathine, betaine, calcium hydroxide, choline, deanol, diethanolamine, diethylamine, 2-(diethylamino)ethanol, ethanolamine, ethylenediamine, N-methylglucamine, hydrabamine, 1H-imidazole, lithium, L-lysine, magnesium, 4-(2-hydroxyethyl)morpholine, piperazine, potassium, 1-(2-hydroxyethyl)pyrrolidine, sodium, triethanolamine, tromethamine, and zinc salts. In certain embodiments, contemplated salts of the invention include, but are not limited to, Na, Ca, K, Mg, Zn or other metal salts. In certain embodiments, contemplated salts of the invention include, but are not limited to, 1-hydroxy-2-naphthoic acid, 2,2-dichloroacetic acid, 2-hydroxyethanesulfonic acid, 2-oxoglutaric acid, 4-acetamidobenzoic acid, 4-aminosalicylic acid, acetic acid, adipic acid, 1-ascorbic acid, l-aspartic acid, benzenesulfonic acid, benzoic acid, (+)-camphoric acid, (+)-camphor-10-sulfonic acid, capric acid (decanoic acid), caproic acid (hexanoic acid), caprylic acid (octanoic acid), carbonic acid, cinnamic acid, citric acid, cyclamic acid, dodecylsulfuric acid, ethane-1,2-disulfonic acid, ethanesulfonic acid, formic acid, fumaric acid, galactaric acid, gentisic acid, d-glucoheptonic acid, d-gluconic acid, d-glucuronic acid, glutamic acid, glutaric acid, glycerophosphoric acid, glycolic acid, hippuric acid, hydrobromic acid, hydrochloric acid, isobutyric acid, lactic acid, lactobionic acid, lauric acid, maleic acid, 1-malic acid, malonic acid, mandelic acid, methanesulfonic acid , naphthalene-1,5-disulfonic acid, naphthalene-2-sulfonic acid, nicotinic acid, nitric acid, oleic acid, oxalic acid, palmitic acid, pamoic acid, phosphoric acid, proprionic acid, 1-pyroglutamic acid, salicylic acid, sebacic acid, stearic acid, succinic acid, sulfuric acid, 1-tartaric acid, thiocyanic acid, p-toluenesulfonic acid, trifluoroacetic acid, and undecylenic acid acid salts.

The pharmaceutically acceptable acid addition salts can also exist as various solvates, such as with water, methanol, ethanol, dimethylformamide, and the like. Mixtures of such solvates can also be prepared. The source of such solvate can be from the solvent of crystallization, inherent in the solvent of preparation or crystallization, or adventitious to such solvent.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically acceptable antioxidants include: (1) water-soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal-chelating agents, such as citric acid, ethylenediamine tetraacetic acid (EDTA), sorbitol, tartaric acid, phosphoric acid, and the like.

### Definitions

Unless otherwise defined herein, scientific and technical terms used in this application shall have the meanings that are commonly understood by those of ordinary skill in the art. Generally, nomenclature used in connection with, and techniques of, chemistry, cell and tissue culture, molecular biology, cell and cancer biology, neurobiology, neurochemistry, virology, immunology, microbiology, pharmacology, genetics and protein and nucleic acid chemistry. described herein, are those well known and commonly used in the art.

The methods and techniques of the present disclosure are generally performed, unless otherwise indicated, according to conventional methods well known in the art and as described in various general and more specific references that are cited and discussed throughout this specification. See, e.g. “Principles of Neural Science”, McGraw-Hill Medical, New York, N.Y. (2000); Motulsky, “Intuitive Biostatistics”, Oxford University Press, Inc. (1995); Lodish et al., “Molecular Cell Biology, 4th ed.”, W. H. Freeman & Co., New York (2000); Griffiths et al., “Introduction to Genetic Analysis, 7th ed.”, W. H. Freeman & Co., N.Y. (1999); and Gilbert et al., “Developmental Biology, 6th ed.”, Sinauer Associates, Inc., Sunderland, MA (2000).

Chemistry terms used herein, unless otherwise defined herein, are used according to conventional usage in the art, as exemplified by “The McGraw-Hill Dictionary of Chemical Terms”. Parker S., Ed., McGraw-Hill, San Francisco, C.A. (1985).

All of the above, and any other publications, patents and published patent applications referred to in this application are specifically incorporated by reference herein. In case of conflict, the present specification, including its specific definitions, will control.

The term “agent” is used herein to denote a chemical compound (such as an organic or inorganic compound, a mixture of chemical compounds), a biological macromolecule (such as a nucleic acid, an antibody, including parts thereof as well as humanized, chimeric and human antibodies and monoclonal antibodies, a protein or portion thereof, e.g., a peptide, a lipid, a carbohydrate), or an extract made from biological materials such as bacteria, plants, fungi, or animal (particularly mammalian) cells or tissues. Agents include, for example, agents whose structure is known, and those whose structure is not known. The ability of such agents to inhibit AR or promote AR degradation may render them suitable as “therapeutic agents” in the methods and compositions of this disclosure.

A “patient,” “subject.” or “individual” are used interchangeably and refer to either a human or a non-human animal. These terms include mammals, such as humans, primates, livestock animals (including bovines, porcines, etc.), companion animals (e.g., canines, felines, etc.) and rodents (e.g., mice and rats).

“Treating” a condition or patient refers to taking steps to obtain beneficial or desired results, including clinical results. Beneficial or desired clinical results can include, but are not limited to, alleviation or amelioration of one or more symptoms or conditions, diminishment of extent of disease, stabilized (i.e. not worsening) state of disease, preventing spread of disease, delay or slowing of disease progression, amelioration or palliation of the disease state, and remission (whether partial or total), whether detectable or undetectable. “Treatment” can also mean prolonging survival as compared to expected survival if not receiving treatment.

The term “preventing” is art-recognized, and when used in relation to a condition, such as a local recurrence (e.g., pain), a disease such as cancer, a syndrome complex such as heart failure or any other medical condition, is well understood in the art, and includes administration of a composition which reduces the frequency of, or delays the onset of, symptoms of a medical condition in a subject relative to a subject which does not receive the composition. Thus, prevention of cancer includes, for example, reducing the number of detectable cancerous growths in a population of patients receiving a prophylactic treatment relative to an untreated control population, and/or delaying the appearance of detectable cancerous growths in a treated population versus an untreated control population, e.g., by a statistically and/or clinically significant amount.

“Administering” or “administration of” a substance, a compound or an agent to a subject can be carried out using one of a variety of methods known to those skilled in the art. For example, a compound or an agent can be administered, intravenously, arterially, intradermally, intramuscularly, intraperitoneally, subcutaneously, ocularly, sublingually, orally (by ingestion), intranasally (by inhalation), intraspinally, intracerebrally, and transdermally (by absorption, e.g., through a skin duct). A compound or agent can also appropriately be introduced by rechargeable or biodegradable polymeric devices or other devices, e.g., patches and pumps, or formulations, which provide for the extended, slow or controlled release of the compound or agent. Administering can also be performed, for example, once, a plurality of times, and/or over one or more extended periods.

Appropriate methods of administering a substance, a compound or an agent to a subject will also depend, for example, on the age and/or the physical condition of the subject and the chemical and biological properties of the compound or agent (e.g., solubility, digestibility, bioavailability, stability and toxicity). In some embodiments, a compound or an agent is administered orally, e.g., to a subject by ingestion. In some embodiments, the orally administered compound or agent is in an extended release or slow release formulation, or administered using a device for such slow or extended release.

As used herein, the phrase “conjoint administration” refers to any form of administration of two or more different therapeutic agents such that the second agent is administered while the previously administered therapeutic agent is still effective in the body (e.g., the two agents are simultaneously effective in the patient, which may include synergistic effects of the two agents). For example, the different therapeutic compounds can be administered either in the same formulation or in separate formulations, either concomitantly or sequentially. Thus, an individual who receives such treatment can benefit from a combined effect of different therapeutic agents.

A “therapeutically effective amount” or a “therapeutically effective dose” of a drug or agent is an amount of a drug or an agent that, when administered to a subject will have the intended therapeutic effect. The full therapeutic effect does not necessarily occur by administration of one dose, and may occur only after administration of a series of doses. Thus, a therapeutically effective amount may be administered in one or more administrations. The precise effective amount needed for a subject will depend upon, for example, the subject's size, health and age, and the nature and extent of the condition being treated, such as cancer or MDS. The skilled worker can readily determine the effective amount for a given situation by routine experimentation.

As used herein, the terms “optional” or “optionally” mean that the subsequently described event or circumstance may occur or may not occur, and that the description includes instances where the event or circumstance occurs as well as instances in which it does not. For example, “optionally substituted alkyl” refers to the alkyl may be substituted as well as where the alkyl is not substituted.

It is understood that substituents and substitution patterns on the compounds of the present invention can be selected by one of ordinary skilled person in the art to result chemically stable compounds which can be readily synthesized by techniques known in the art, as well as those methods set forth below, from readily available starting materials. If a substituent is itself substituted with more than one group, it is understood that these multiple groups may be on the same carbon or on different carbons, so long as a stable structure results.

As used herein, the term “optionally substituted” refers to the replacement of one to six hydrogen radicals in a given structure with the radical of a specified substituent including, but not limited to: hydroxyl, hydroxyalkyl, alkoxy, halogen, alkyl, nitro, silyl, acyl, acyloxy, aryl, cycloalkyl, heterocyclyl, amino, aminoalkyl, cyano, haloalkyl, haloalkoxy, —OCO—CH₂—O-alkyl, —OP(O)(O-alkyl)₂ or —CH2—OP(O)(O-alkyl)₂. Preferably, “optionally substituted” refers to the replacement of one to four hydrogen radicals in a given structure with the substituents mentioned above. More preferably, one to three hydrogen radicals are replaced by the substituents as mentioned above. It is understood that the substituent can be further substituted.

As used herein, the term “alkyl” refers to saturated aliphatic groups, including but not limited to C₁-C₁₀ straight-chain alkyl groups or C₁-C₁₀ branched-chain alkyl groups. Preferably, the “alkyl” group refers to C1-C6 straight-chain alkyl groups or C₁-C₆ branched-chain alkyl groups. Most preferably, the “alkyl” group refers to C₁-C₄ straight-chain alkyl groups or C₁-C₄ branched-chain alkyl groups. Examples of “alkyl” include, but are not limited to, methyl, ethyl, 1-propyl, 2-propyl, n-butyl, sec-butyl, tert-butyl, 1-pentyl, 2-pentyl, 3-pentyl, neo-pentyl, 1-hexyl, 2-hexyl, 3-hexyl, 1-heptyl, 2-heptyl, 3-heptyl, 4-heptyl, 1-octyl, 2-octyl, 3-octyl or 4-octyl and the like. The “alkyl” group may be optionally substituted.

The term “acyl” is art-recognized and refers to a group represented by the general formula hydrocarbylC(O)—, preferably alkylC(O)—.

The term “acylamino” is art-recognized and refers to an amino group substituted with an acyl group and may be represented, for example, by the formula hydrocarbylC(O)NH—.

The term “acyloxy” is art-recognized and refers to a group represented by the general formula hydrocarbylC(O)O—, preferably alkylC(O)O—.

The term “alkoxy” refers to an alkyl group having an oxygen attached thereto. Representative alkoxy groups include methoxy, ethoxy, propoxy, tert-butoxy and the like.

The term “alkoxyalkyl” refers to an alkyl group substituted with an alkoxy group and may be represented by the general formula alkyl-O-alkyl.

The term “alkyl” refers to saturated aliphatic groups, including straight-chain alkyl groups, branched-chain alkyl groups, cycloalkyl (alicyclic) groups, alkyl-substituted cycloalkyl groups, and cycloalkyl-substituted alkyl groups. In preferred embodiments, a straight chain or branched chain alkyl has 30 or fewer carbon atoms in its backbone (e.g., C₁₋₃₀ for straight chains, C₃₋₃₀ for branched chains), and more preferably 20 or fewer.

Moreover, the term “alkyl” as used throughout the specification, examples, and claims is intended to include both unsubstituted and substituted alkyl groups, the latter of which refers to alkyl moieties having substituents replacing a hydrogen on one or more carbons of the hydrocarbon backbone, including haloalkyl groups such as trifluoromethyl and 2,2,2-trifluoroethyl, etc.

The term “C_{x-y}” or “C_{x-y}”, when used in conjunction with a chemical moiety, such as, acyl, acyloxy, alkyl, alkenyl, alkynyl, or alkoxy is meant to include groups that contain from x to y carbons in the chain. Coalkyl indicates a hydrogen where the group is in a terminal position, a bond if internal. A C₁₋₆alkyl group, for example, contains from one to six carbon atoms in the chain.

The term “alkylamino”, as used herein, refers to an amino group substituted with at least one alkyl group.

The term “alkylthio”, as used herein, refers to a thiol group substituted with an alkyl group and may be represented by the general formula alkyl S—.

The term “amide”, as used herein, refers to a group

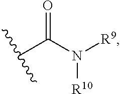

wherein R⁹ and R¹⁰ each independently represent a hydrogen or hydrocarbyl group, or R⁹ and R¹⁰ taken together with the N atom to which they are attached complete a heterocycle having from 4 to 8 atoms in the ring structure.

The terms “amine” and “amino” are art-recognized and refer to both unsubstituted and substituted amines and salts thereof, e.g., a moiety that can be represented by

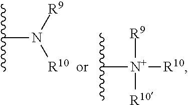

wherein R⁹, R¹⁰, and R¹⁰, each independently represent a hydrogen or a hydrocarbyl group, or R⁹ and R¹⁰ taken together with the N atom to which they are attached complete a heterocycle having from 4 to 8 atoms in the ring structure.

The term “aminoalkyl”, as used herein, refers to an alkyl group substituted with an amino group.

The term “aralkyl”, as used herein, refers to an alkyl group substituted with an aryl group.

The term “aryl” as used herein include substituted or unsubstituted single-ring aromatic groups in which each atom of the ring is carbon. Preferably the ring is a 5- to 7-membered ring, more preferably a 6-membered ring. The term “aryl” also includes polycyclic ring systems having two or more cyclic rings in which two or more carbons are common to two adjoining rings wherein at least one of the rings is aromatic, e.g., the other cyclic rings can be cycloalkyls, cycloalkenyls, cycloalkynyls, aryls, heteroaryls, and/or heterocyclyls. Aryl groups include benzene, naphthalene, phenanthrene, phenol, aniline, and the like.

The term “carbamate” is art-recognized and refers to a group

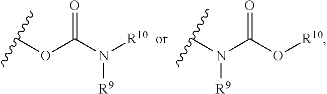

wherein R⁹ and R¹⁰ independently represent hydrogen or a hydrocarbyl group.

The term “carbocyclylalkyl”, as used herein, refers to an alkyl group substituted with a carbocycle group.

The term “carbocycle” includes 5-7 membered monocyclic and 8-12 membered bicyclic rings. Each ring of a bicyclic carbocycle may be selected from saturated, unsaturated and aromatic rings. Carbocycle includes bicyclic molecules in which one, two or three or more atoms are shared between the two rings. The term “fused carbocycle” refers to a bicyclic carbocycle in which each of the rings shares two adjacent atoms with the other ring. Each ring of a fused carbocycle may be selected from saturated, unsaturated and aromatic rings. In an exemplary embodiment, an aromatic ring, e.g., phenyl, may be fused to a saturated or unsaturated ring, e.g., cyclohexane, cyclopentane, or cyclohexene. Any combination of saturated, unsaturated and aromatic bicyclic rings, as valence permits, is included in the definition of carbocyclic. Exemplary “carbocycles” include cyclopentane, cyclohexane, bicyclo[2.2.1]heptane, 1,5-cyclooctadiene, 1,2,3,4-tetrahydronaphthalene, bicyclo[4.2.0]oct-3-ene, naphthalene and adamantane. Exemplary fused carbocycles include decalin, naphthalene, 1,2,3,4-tetrahydronaphthalene, bicyclo[4.2.0]octane, 4,5,6,7-tetrahydro-1H-indene and bicyclo[4.1.0]hept-3-ene. “Carbocycles” may be substituted at any one or more positions capable of bearing a hydrogen atom.

The term “carbocyclylalkyl”, as used herein, refers to an alkyl group substituted with a carbocycle group.

The term “carbonate” is art-recognized and refers to a group —OCO₂—.

The term “carboxy”, as used herein, refers to a group represented by the formula —CO₂H.

The term “cycloalkyl” includes substituted or unsubstituted non-aromatic single ring structures, preferably 4- to 8-membered rings, more preferably 4- to 6-membered rings. The term “cycloalkyl” also includes polycyclic ring systems having two or more cyclic rings in which two or more carbons are common to two adjoining rings wherein at least one of the rings is cycloalkyl and the substituent (e.g., R¹⁰⁰) is attached to the cycloalkyl ring, e.g., the other cyclic rings can be cycloalkyls, cycloalkenyls, cycloalkynyls, aryls, heteroaryls, and/or heterocyclyls. Heteroaryl groups include, for example, pyrrole, furan, thiophene, imidazole, oxazole, thiazole, pyrazole, pyridine, pyrazine, pyridazine, pyrimidine, denzodioxane, tetrahydroquinoline, and the like.

The term “ester”, as used herein, refers to a group —C(O)OR⁹ wherein R⁹ represents a hydrocarbyl group.

The term “ether”, as used herein, refers to a hydrocarbyl group linked through an oxygen to another hydrocarbyl group. Accordingly, an ether substituent of a hydrocarbyl group may be hydrocarbyl-O—. Ethers may be either symmetrical or unsymmetrical. Examples of ethers include, but are not limited to, heterocycle-O-heterocycle and aryl-O-heterocycle. Ethers include “alkoxyalkyl” groups, which may be represented by the general formula alkyl-O-alkyl.

The terms “halo” and “halogen” as used herein means halogen and includes chloro, fluoro, bromo, and iodo.

The terms “hetaralkyl” and “heteroaralkyl”, as used herein, refers to an alkyl group substituted with a hetaryl group.

The terms “heteroaryl” and “hetaryl” include substituted or unsubstituted aromatic single ring structures, preferably 5- to 7-membered rings, more preferably 5- to 6-membered rings, whose ring structures include at least one heteroatom, preferably one to four heteroatoms, more preferably one or two heteroatoms. The terms “heteroaryl” and “hetaryl” also include polycyclic ring systems having two or more cyclic rings in which two or more carbons are common to two adjoining rings wherein at least one of the rings is heteroaromatic, e.g., the other cyclic rings can be cycloalkyls, cycloalkenyls, cycloalkynyls, aryls, heteroaryls, and/or heterocyclyls. Heteroaryl groups include, for example, pyrrole, furan, thiophene, imidazole, oxazole, thiazole, pyrazole, pyridine, pyrazine, pyridazine, and pyrimidine, and the like.

The term “heteroatom” as used herein means an atom of any element other than carbon or hydrogen. Preferred heteroatoms are nitrogen, oxygen, and sulfur.

The term “heterocyclylalkyl”, as used herein, refers to an alkyl group substituted with a heterocycle group.

The terms “heterocyclyl”, “heterocycle”, and “heterocyclic” refer to substituted or unsubstituted non-aromatic ring structures, preferably 3- to 10-membered rings, more preferably 3- to 7-membered rings, whose ring structures include at least one heteroatom, preferably one to four heteroatoms, more preferably one or two heteroatoms. The terms “heterocyclyl” and “heterocyclic” also include polycyclic ring systems having two or more cyclic rings in which two or more carbons are common to two adjoining rings wherein at least one of the rings is heterocyclic, e.g., the other cyclic rings can be cycloalkyls, cycloalkenyls, cycloalkynyls, aryls, heteroaryls, and/or heterocyclyls. Heterocyclyl groups include, for example, piperidine, piperazine, pyrrolidine, morpholine, lactones, lactams, and the like.

The term “hydrocarbyl”, as used herein, refers to a group that is bonded through a carbon atom that does not have a ═O or ═S substituent, and typically has at least one carbon-hydrogen bond and a primarily carbon backbone, but may optionally include heteroatoms. Thus, groups like methyl, ethoxyethyl, 2-pyridyl, and even trifluoromethyl are considered to be hydrocarbyl for the purposes of this application, but substituents such as acetyl (which has a ═O substituent on the linking carbon) and ethoxy (which is linked through oxygen, not carbon) are not. Hydrocarbyl groups include, but are not limited to aryl, heteroaryl, carbocycle, heterocycle, alkyl, alkenyl, alkynyl, and combinations thereof.

The term “hydroxyalkyl”, as used herein, refers to an alkyl group substituted with a hydroxy group.

The term “lower” when used in conjunction with a chemical moiety, such as, acyl, acyloxy, alkyl, alkenyl, alkynyl, or alkoxy is meant to include groups where there are ten or fewer atoms in the substituent, preferably six or fewer. A “lower alkyl”, for example, refers to an alkyl group that contains ten or fewer carbon atoms, preferably six or fewer. In certain embodiments, acyl, acyloxy, alkyl, alkenyl, alkynyl, or alkoxy substituents defined herein are respectively lower acyl, lower acyloxy, lower alkyl, lower alkenyl, lower alkynyl, or lower alkoxy, whether they appear alone or in combination with other substituents, such as in the recitations hydroxyalkyl and aralkyl (in which case, for example, the atoms within the aryl group are not counted when counting the carbon atoms in the alkyl substituent).

The terms “polycyclyl”, “polycycle”, and “polycyclic” refer to two or more rings (e.g., cycloalkyls, cycloalkenyls, cycloalkynyls, aryls, heteroaryls, and/or heterocyclyls) in which two or more atoms are common to two adjoining rings, e.g., the rings are “fused rings”. Each of the rings of the polycycle can be substituted or unsubstituted. In certain embodiments, each ring of the polycycle contains from 3 to 10 atoms in the ring, preferably from 5 to 7.

The term “sulfate” is art-recognized and refers to the group —OSO₃H, or a pharmaceutically acceptable salt thereof.

The term “sulfonamide” is art-recognized and refers to the group represented by the general formulae

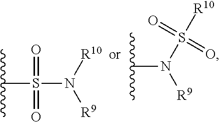

wherein R⁹ and R¹⁰ independently represents hydrogen or hydrocarbyl.

The term “sulfoxide” is art-recognized and refers to the group —S(O)—.

The term “sulfonate” is art-recognized and refers to the group SO₃H, or a pharmaceutically acceptable salt thereof.

The term “sulfone” is art-recognized and refers to the group —S(O)₂—.

The term “substituted” refers to moieties having substituents replacing a hydrogen on one or more carbons of the backbone. It will be understood that “substitution” or “substituted with” includes the implicit proviso that such substitution is in accordance with permitted valence of the substituted atom and the substituent, and that the substitution results in a stable compound, e.g., which does not spontaneously undergo transformation such as by rearrangement, cyclization, elimination, etc. As used herein, the term “substituted” is contemplated to include all permissible substituents of organic compounds. In a broad aspect, the permissible substituents include acyclic and cyclic, branched and unbranched, carbocyclic and heterocyclic, aromatic and non-aromatic substituents of organic compounds. The permissible substituents can be one or more and the same or different for appropriate organic compounds. For purposes of this invention, the heteroatoms such as nitrogen may have hydrogen substituents and/or any permissible substituents of organic compounds described herein which satisfy the valences of the heteroatoms. Substituents can include any substituents described herein, for example, a halogen, a hydroxyl, a carbonyl (such as a carboxyl, an alkoxycarbonyl, a formyl, or an acyl), a thiocarbonyl (such as a thioester, a thioacetate, or a thioformate), an alkoxyl, a phosphoryl, a phosphate, a phosphonate, a phosphinate, an amino, an amido, an amidine, an imine, a cyano, a nitro, an azido, a sulfhydryl, an alkylthio, a sulfate, a sulfonate, a sulfamoyl, a sulfonamido, a sulfonyl, a heterocyclyl, an aralkyl, or an aromatic or heteroaromatic moiety. It will be understood by those skilled in the art that the moieties substituted on the hydrocarbon chain can themselves be substituted, if appropriate.

The term “thioalkyl”, as used herein, refers to an alkyl group substituted with a thiol group.

The term “thioester”, as used herein, refers to a group —C(O)SR⁹ or —SC(O)R⁹ wherein R⁹ represents a hydrocarbyl.

The term “thioether”, as used herein, is equivalent to an ether, wherein the oxygen is replaced with a sulfur.

The term “urea” is art-recognized and may be represented by the general formula

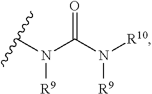

wherein R⁹ and R¹⁰ independently represent hydrogen or a hydrocarbyl.

The term “modulate” as used herein includes the inhibition or suppression of a function or activity (such as cell proliferation) as well as the enhancement of a function or activity.

The phrase “pharmaceutically acceptable” is art-recognized. In certain embodiments, the term includes compositions, excipients, adjuvants, polymers and other materials and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

“Pharmaceutically acceptable salt” or “salt” is used herein to refer to an acid addition salt or a basic addition salt which is suitable for or compatible with the treatment of patients.

The term “pharmaceutically acceptable acid addition salt” as used herein means any non-toxic organic or inorganic salt of any base compounds represented by Formula I. Illustrative inorganic acids which form suitable salts include hydrochloric, hydrobromic, sulfuric and phosphoric acids, as well as metal salts such as sodium monohydrogen orthophosphate and potassium hydrogen sulfate. Illustrative organic acids that form suitable salts include mono-, di-, and tricarboxylic acids such as glycolic, lactic, pyruvic, malonic, succinic, glutaric, fumaric, malic, tartaric, citric, ascorbic, maleic, benzoic, phenylacetic, cinnamic and salicylic acids, as well as sulfonic acids such as p-toluene sulfonic and methanesulfonic acids. Either the mono or di-acid salts can be formed, and such salts may exist in either a hydrated, solvated or substantially anhydrous form. In general, the acid addition salts of compounds of Formula I are more soluble in water and various hydrophilic organic solvents, and generally demonstrate higher melting points in comparison to their free base forms. The selection of the appropriate salt will be known to one skilled in the art. Other non-pharmaceutically acceptable salts, e.g., oxalates, may be used, for example, in the isolation of compounds of Formula I for laboratory use, or for subsequent conversion to a pharmaceutically acceptable acid addition salt.

The term “pharmaceutically acceptable basic addition salt” as used herein means any non-toxic organic or inorganic base addition salt of any acid compounds represented by Formula I or any of their intermediates. Illustrative inorganic bases which form suitable salts include lithium, sodium, potassium, calcium, magnesium, or barium hydroxide. Illustrative organic bases which form suitable salts include aliphatic, alicyclic, or aromatic organic amines such as methylamine, trimethylamine and picoline or ammonia. The selection of the appropriate salt will be known to a person skilled in the art.

Many of the compounds useful in the methods and compositions of this disclosure have at least one stereogenic center in their structure. This stereogenic center may be present in a R or a S configuration, said R and S notation is used in correspondence with the rules described in Pure Appl. Chem. (1976), 45, 11-30. The disclosure contemplates all stereoisomeric forms such as enantiomeric and diastereoisomeric forms of the compounds, salts, prodrugs or mixtures thereof (including all possible mixtures of stereoisomers). See, e.g., WO 01/062726.

Furthermore, certain compounds which contain alkenyl groups may exist as Z (zusammen) or E (entgegen) isomers. In each instance, the disclosure includes both mixture and separate individual isomers.

Some of the compounds may also exist in tautomeric forms. Such forms, although not explicitly indicated in the formulae described herein, are intended to be included within the scope of the present disclosure.

“Prodrug” or “pharmaceutically acceptable prodrug” refers to a compound that is metabolized, for example hydrolyzed or oxidized, in the host after administration to form the compound of the present disclosure (e.g., compounds of formula I). Typical examples of prodrugs include compounds that have biologically labile or cleavable (protecting) groups on a functional moiety of the active compound. Prodrugs include compounds that can be oxidized, reduced, aminated, deaminated, hydroxylated, dehydroxylated, hydrolyzed, dehydrolyzed, alkylated, dealkylated, acylated, deacylated, phosphorylated, or dephosphorylated to produce the active compound. Examples of prodrugs using ester or phosphoramidate as biologically labile or cleavable (protecting) groups are disclosed in U.S. Pat. Nos. 6,875,751, 7,585,851, and 7,964,580, the disclosures of which are incorporated herein by reference. The prodrugs of this disclosure are metabolized to produce a compound of Formula I. The present disclosure includes within its scope, prodrugs of the compounds described herein. Conventional procedures for the selection and preparation of suitable prodrugs are described, for example, in “Design of Prodrugs” Ed. H. Bundgaard, Elsevier, 1985.

The phrase “pharmaceutically acceptable carrier” as used herein means a pharmaceutically acceptable material, composition or vehicle, such as a liquid or solid filter, diluent, excipient, solvent or encapsulating material useful for formulating a drug for medicinal or therapeutic use.

The term “Log of solubility”, “LogS” or “logS” as used herein is used in the art to quantify the aqueous solubility of a compound. The aqueous solubility of a compound significantly affects its absorption and distribution characteristics. A low solubility often goes along with a poor absorption. LogS value is a unit stripped logarithm (base 10) of the solubility measured in mol/liter.

### EXAMPLES

The invention now being generally described, it will be more readily understood by reference to the following examples which are included merely for purposes of illustration of certain aspects and embodiments of the present invention, and are not intended to limit the invention.

### Example 1: Preparation of Exemplary Compounds of the Disclosure

Exemplary compounds of the disclosure can be synthesized using methods analogous to those set forth in Scheme 1.

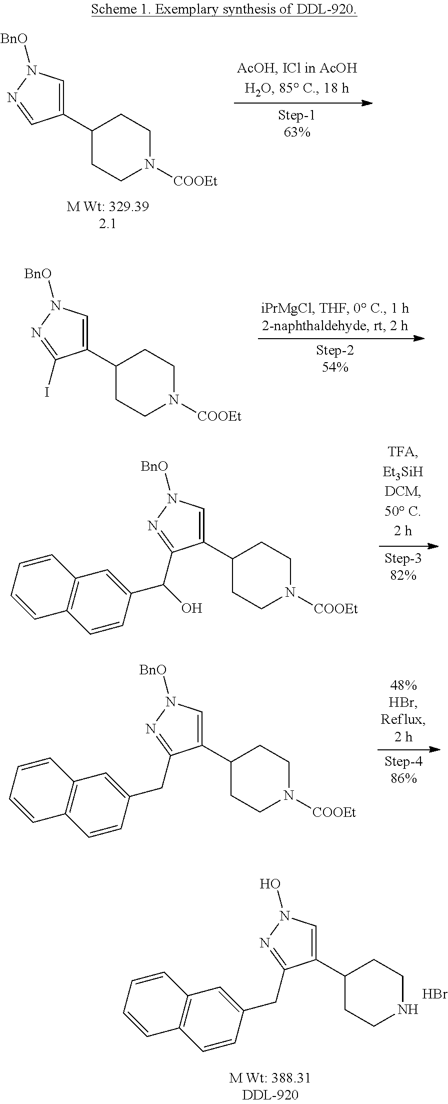 [SMILES: CCOC(=O)N1CCC(c2cnn(OCc3ccccc3)c2)CC1 ; CCOC(=O)N1CCC(c2cn(OCc3ccccc3)nc2I)CC1 ; CCOC(=O)N1CCC(c2cn(OCc3ccccc3)nc2C(O)c2ccc3ccccc3c2)CC1 ; CCOC(=O)N1CCC(c2cn(OCc3ccccc3)nc2Cc2ccc3ccccc3c2)CC1 ; On1cc(C2CCNCC2)c(Cc2ccc3ccccc3c2)n1 ; Br]

Ethyl 4-(1-(benzyloxy)-3-iodo-1H-pyrazol-4-yl)piperidine-1-carboxylate (2.2). Step-1: To a solution of compound-2.1 (1.435 g, 4.36 mmol) in AcOH (7.25 mL) was added a solution of ICI (848.7 mg, 5.23 mmol) in AcOH (7.25 mL) followed by water (21.7 mL) and the resulting reaction mixture was stirred at 85° C.for 18 h. The progress of the reaction was monitored by TLC and after completion of the reaction, the reaction mixture was cooled and quenched with Na₂S₂O₃ solution (0.1 M, 20 mL). Water (30 mL) was added and extracted with Et₂O(3×45 mL), dried over anhydrous sodium sulfate, filtered, and concentrated in vacuo. The residue was purified by prepacked silica gel column (24 g), eluted with ethyl acetate in hexanes (0 to 20%). The desired fractions (20%) were concentrated and dried to yield compound-2.2 (1.261 g, 63%) as colorless solid. ¹H NMR (300 MHZ, CHLOROFORM-d) δ=7.44-7.31 (m, 3H), 7.30-7.23 (m, 2H), 6.56 (s, 1H), 5.25 (s, 2H), 4.29-4.05 (m, 4H), 2.80 (br t, J=12.6 Hz, 2H), 2.39 (tt, J=3.6, 11.9 Hz, 1H), 1.77 (br d, J=12.3 Hz, 2H), 1.38-1.11 (m, 5H).

Ethyl 4-(1-(benzyloxy)-3-(hydroxy(naphthalen-2-yl)methyl)-1H-pyrazol-4-yl)piperidine-1-carboxylate (2.3). Step-2: Compound-2.2 (500 mg, 1.10 mmol) was dissolved in dry THF (8.0 mL), cooled to 0° C. and iPrMgCl (0.824 mL, 2.0 M in THF, 1.5 cq) was added and the resulting reaction mixture was stirred for 1 h. Then, a solution of 2-naphthaldehyde (317 mg, 2.03 mmol) in THF (4.0 mL) was added and the mixture was allowed to slowly reach room temperature overnight. The reaction was quenched with saturated NH₄Cl and water (1:1, 15 mL), extracted with Et₂O (3×20 mL), dried over anhydrous sodium sulfate, filtered and concentrated. The residue was purified by prepacked silica gel column (24 g), eluted with ethyl acetate in hexanes (0 to 50%). The desired fractions (50%) were concentrated and dried provided desired compound-2.3 (290 mg, 54%) as colorless solid. (+csi)[ M+H]⁺=486.6; 1H NMR (300 MHZ, CHLOROFORM-d) δ=7.91-7.76 (m, 4H), 7.54-7.42 (m, 3H), 7.41-7.27 (m, 5H), 6.69 (s, 1H), 6.02 (d, J=4.7 Hz, 1H), 5.27 (s, 2H), 4.10-3.85 (m, 4H), 3.07 (br d, J=5.3 Hz, 1H), 2.56-2.41 (m, 2H), 2.40-2.27 (m, 1H), 1.48-1.30 (m, 2H), 1.20 (t, J=6.7 Hz, 3H), 1.13-0.98 (m, 2H).

Ethyl 4-(1-(benzyloxy)-3-(naphthalen-2-ylmethyl)-1H-pyrazol-4-yl)piperidine-1-carboxylate (2.4). Step-3: To a solution of compound-2.3 (286.7 mg, 590.4 umol) in dry CH₂Cl₂ (20 mL) was added Et₃SiH (189 μL, 1.181 mmol) followed by the addition of trifluoroacetic acid (1.36 mL, 17.71 mmol) under N₂ at 0° C. The resulting reaction mixture was heated to reflux at 50 ° C. for 2 h. The progress of the reaction was monitored by TLC and after completion of the reaction, the mixture was cooled to 0° C. and water (20 mL) was added to the reaction mixture followed by extraction with Et₂O (3×10 mL). The combined ether phases were washed with saturated NaHCO₃ until pH 7 was obtained. The organic phases were washed with brine (5 mL), dried over anhydrous sodium sulfate, filtered and concentrated. The residue was purified by prepacked silica gel column (24 g), eluted with ethyl acetate in hexanes (0 to 30%). The desired fractions (25 to 28%) were concentrated and dried to provide compound-2.4 (226.9 mg, 82%) as colorless liquid. (+csi)[M+H]⁺=470.6; ¹H NMR (300 MHZ, CHLOROFORM-d) δ=7.86-7.70 (m, 3H), 7.60 (s, 1H), 7.51-7.40 (m, 2H), 7.38-7.26 (m, 6H), 6.69 (s, 1H), 5.27 (s, 2H), 4.15-3.95 (m, 6H), 2.69-2.49 (m, 2H), 2.45 -2.30 (m, 1H), 1.49 (br d, J=11.7 Hz, 2H), 1.25-1.02 (m, 5H).

3-(naphthalen-2-ylmethyl)-4-(piperidin-4-yl)-1H-pyrazol-1-ol (DDL-920). Step-4: Compound-2.4 (88 mg, 190 μmol) and concentrated aqueous HCl (6.33 mL) were heated to 130° C. and stirred for 1 h. The progress of the reaction was monitored by LCMS and indicated complete conversion to product. The excess HCl was evaporated and recrystallized from MeOH/diethyl ether, filtered and the solid was dried under high vacuum. The solid was finally triturated with diethyl ether, decanted the solvent, and dried under high vacuum to yield VLCH-0020330 (55.3 mg, 86%) as a colorless solid with >95% purity. (+esi)[M+H]⁺=308.3 (freebase); ¹H NMR (300 MHZ, METHANOL-d4) δ=7.84-7.73 (m, 3H), 7.67 (s, 1H), 7.48 (s, 1H), 7.46-7.39 (m, 2H), 7.35 (dd, J=1.5, 8.5 Hz, 1H), 4.13 (s, 2H), 3.30-3.21 (m, 3H), 3.00-2.85 (m, 2H), 2.84-2.71 (m, 1H), 1.86-1.75 (m, 2H), 1.73-1.53 (m, 2H).

Compounds related to DDL-920 may be synthesissed via analogous methods, for example by the method set forth in Scheme 2.

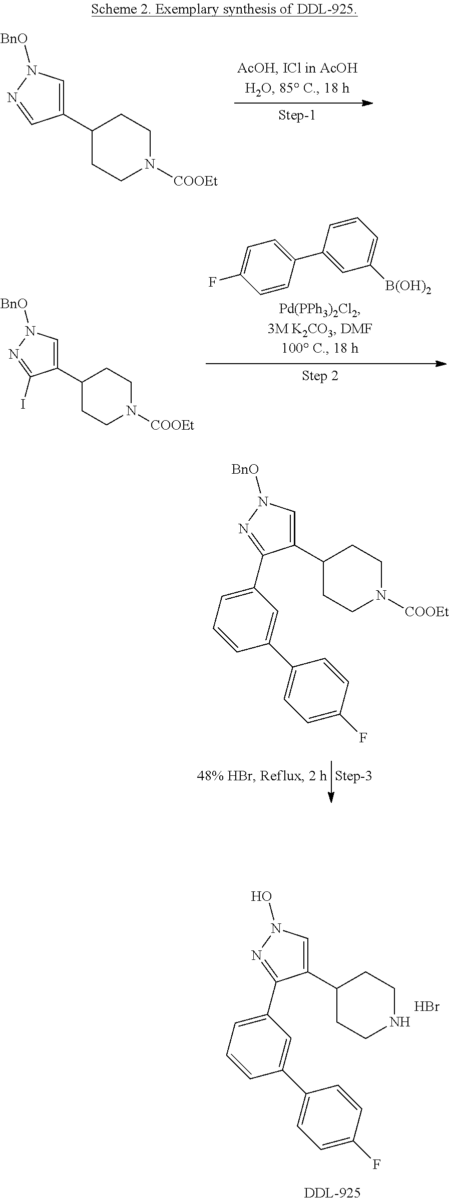 [SMILES: CCOC(=O)N1CCC(c2cnn(OCc3ccccc3)c2)CC1 ; OB(O)c1cccc(-c2ccc(F)cc2)c1 ; CCOC(=O)N1CCC(c2cn(OCc3ccccc3)nc2I)CC1 ; CCOC(=O)N1CCC(c2cn(OCc3ccccc3)nc2-c2cccc(-c3ccc(F)cc3)c2)CC1 ; On1cc(C2CCNCC2)c(-c2cccc(-c3ccc(F)cc3)c2)n1 ; Br]

### Example 2: Biological Testing of Exemplary Compounds of the Disclosure

Tonic currents mediated by a1B28 GABA_{A}Rs in fluorescently identified PV+INs and γ-oscillations in the CA3 region hippocampal slices will be recorded. The assays will serve to identify compounds with an ≥0.7 blockin3g efficacy (EC₇₀) of the tonic conductance at ≤100 nM, a ≥2-fold specificity over phasic inhibition, and a ≥2-fold change at ≤100 nM in the power of γ-oscillations measured between 30-120 Hz.

### Sample Preparation

Mice aged 8-24 weeks of both sexes will be anesthetized with halothane and decapitated according to the UCLA Chancellor's Animal Research Committee protocol. Horizontal 350 μm hippocampal slices are cut on a Leica VT1000S Vibratome in ice-cold N-Methyl-D-Glucamine (NMDG)-based HEPES-buffered cutting solution, containing in mM: NMDG 135, D-glucose 10, MgCl₂ 4, CaCl₂0.5, KCl 1, KH₂PO₄ 1.2, HEPES 26, kynurenic acid 3, pH 7.3-7.4, 295-297 mOsm/l, bubbled with 100% O₂. Slices will then be incubated for 30 min at room temperature (˜22° C.) in an interface chamber in modified sucrose aCSF, containing in mM: NaCl 85, D-glucose 25, sucrose 55, KCl 2.5, NaH₂PO₄ 1.25, CaCl₂ 0.5, MgCl₂ 4, NaHCO₃ 26, at pH 7.3-7.4 when bubbled with 95% O₂, 5% CO₂. Over the next 30 min the temperature will be gradually increased to 32° C. and the sucrose aCSF will be gradually substituted over the same time period for normal aCSF, containing in mM: NaCl 126, D-glucose 10, MgCl₂ 2, CaCl₂ 2, KCl 2.5, NaH₂PO₄ 1.25, Na Pyruvate 1.5, L-Glutamine 1, NaHCO₃ 26, pH 7.3-7.4 when bubbled with 95% O_{2,} 5% CO₂. Healthy slices can remain under these storage conditions for up to 6 hours.

### Tonic and Phasic Current Recordings in Fluorescently Labeled PV+INs.

For these experiments mice expressing hChR2(H134R)-tdTomato specifically in PV+Ins will be used. The expression of tdTomato will be particularly useful to identify PV INs in brain slices for subsequent electrophysiological recordings. This protein is 6-times brighter than EGFP, and its peak excitation wavelength (554 nm) lies outside the excitation spectrum of ChR2 (473 nm); thus ChR2 will not be stimulated while the neurons are being identified through tdTomato excitation by a 561 nm yellow laser. Whole-cell recordings will be done at 35±1° C. in fluorescently identified cortical and hippocampal PV+INs in normal aCSF supplemented with 5 μM GABA to ensure the uniform activation of δ-GABA_{A}Rs. The pipette solution is composed of (in mM): 140 Cs-met, 2 MgCl₂ 10 HEPES, 0.2 EGTA, 2 Na₂-ATP, 0.2 Na₂-GTP. The pH is adjusted to 7.2 with CsOH and its osmolarity is 285-290 mOsm. Cells will be voltage-clamped at 0 mV where tonic and phasic GABA_{A}R mediated currents can be recorded without interference from excitatory events. The tonic and phasic components of the currents will be determined as published using Gaussian fits to the all-points histograms of 30 s epochs of recordings during the pre-drug period, 5-10 min after the washin of the drug to be tested, and 1-2 min after washing-in 40 μM of the pan-GABA_{A}R blocker gabazine (SR-95531). The magnitude of the tonic and phasic currents willbe determined by subtracting the currents in the presence of gabazine. The tonic currents during control conditions will also be normalized by whole-cell capacitance (a measure of cell size).

### Specificity of the Compounds for Tonic vs Phasic Inhibition

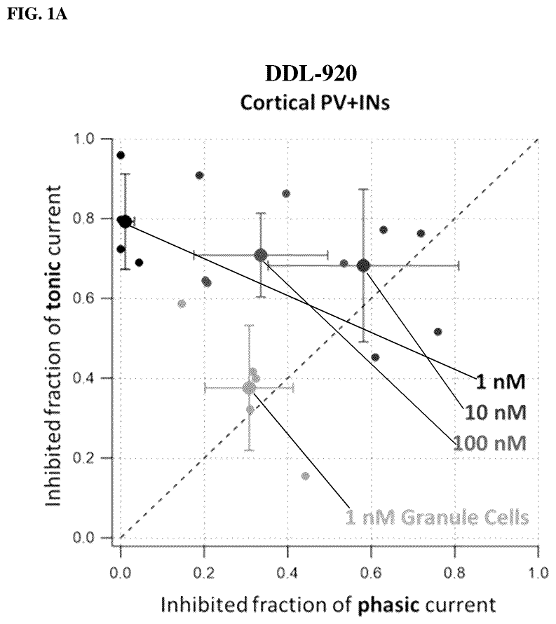

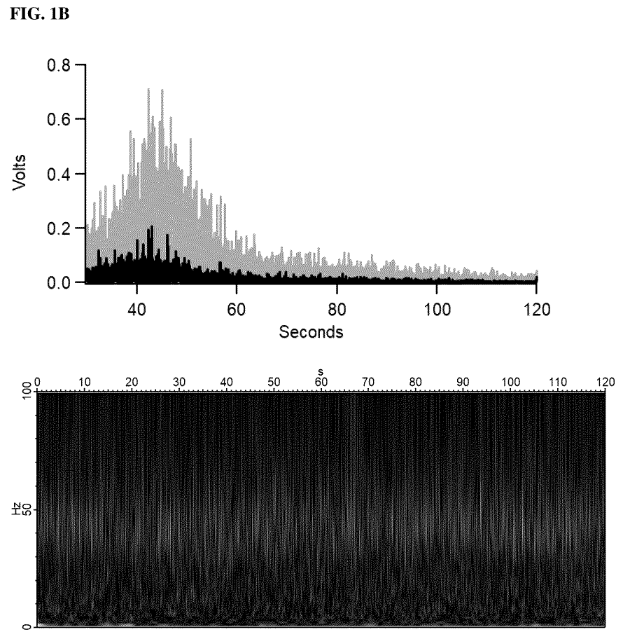

As tonic inhibition in PV+INs is mediated by extrasynaptic δ-GABA_{A}Rs, while phasic inhibition is generated by fast synaptic α1β2γ2 receptors, a differential effect on the tonic and phasic currents will indicate how specific a given compound is on the native α1β2δ receptors of PV+INs. Specificity plots can be obtained by graphing the fraction inhibited by the investigated compound of the tonic current vs the fraction inhibited of the phasic current (FIG. 1A). The two fractions are calculated as: 1—(current in drug/current in control). The specificity for the inhibition of the tonic vs phasic current is calculated as: specificity for tonic/specificity for phasic. For 1 nM DDL-920 (FIG. 1A) this value is: 0.792/0.011=72.

### Effects of Compounds on In Vitro γ-Oscillations in the CA3 Region Hippocampal Slices

The oscillations can be recorded in the presence of 50 nM kainic acid in horizontally cut hippocampal slices just below the CA3 pyramidal cells layer, and the oscillations recorded in vitro correspond well to those recorded in vivo during spatial reference memory. In this region, the only cells with δ-GABA_{A}Rs are the PV+INs. The squared magnitude of the FFT of the oscillations will be analyzed between 30-120 Hz. The area under the FFT curve will be calculated between these two frequencies and compared during a 2 min epoch just before the perfusion of the drug to the last 2 min of a 10 min drug perfusion. The ratios of the areas in-drug/pre-drug will be calculated as in FIG. 1B.

### Measuring the Selectivity of the Compounds for α1, α4, and α6 and α6 Subunits Present in Various δ0 Subunit Containing GABAAR found in the Brain

In addition to the α1β2δ GABAARs of PV+INs, the following native δ-GABA_{A}Rs subunit combinations are present in the brain: (α6β2δ on cerebellar granule cells; α4β2/3δ are most prominent in the forebrain where they are found among other neurons on dentate gyrus granule cells. To establish selectivity of the compounds for the α1β2δ GABA_{A}Rs of PV+INs, the compounds effects in cerebellar granule cells (α6β2δ) and dentate gyrus granule cells (α4β2/3δ) will be compared.

Selectivity measurement will be performed by dividing the blocking fractions of the compounds for tonic inhibition obtained for the PV+IN above by the values determined for cerebellar and dentate gyrus granule cells. As shown in FIG. 1A, the value for 1 nM DDL-920 is: 0.792/0.376 =2.11 for the PV+IN dentate granule cell comparison.

### SA 3.2: Target Engagement, PD, and Efficacy Assessment by LFP Recordings In Vivo

Video observation of the mouse's activity will be performed with a top-mounted infrared USB camera. The software used for the recording (iSpy) calculates in real time the deviations (in %) between consecutive frames and generates a text file with time-stamped information and the deviation values. Local field potential recordings will be performed with a custom-made miniature dual headstage amplifier connected to the electrode sockets and wired to an electrical commutator (Pinnacle Technology Inc.). The signals are then lead to a 16 channel extracellular amplifier (A-M Systems; 1,000 gain), and are sampled at 2,048 s⁻¹ using a NI data acquisition board. Data acquisition is done using Igor NIDaq tool (Wavemetrics, Lake Oswego, OR). Mouse activity graphs and corresponding local field potentials are aligned using a custom Igor64 software. Mice rapidly habituate to the setup due to the lightweight headstage and the flexible custom-made cables that allow several months of continuous recordings.

### LFP Analysis for Target Engagement, PD, and In Vivo Efficacy

LFP recorded at a high bandwidth (0.1-1,000 Hz) and mouse activity levels derived from the video recordings will identify periods of sleep/inactivity and movement. The LFP recordings can be used to detect awake and sleep periods (NREM and REM) based on the relationship between oscillatory patterns and on the mouse's movement/activity stages derived from the video recordings. The main analysis will focus on detecting oscillations in the θ, low and high γ range. γ-oscillation power and frequency will be measured at the exact time points used for the PK analysis after SQ injections. Changes in power will be assessed as described for the in vitro oscillations. Thus, a reading of the target engagement and PD properties of the compounds will be obtained. According to existing data data in female mice during the ovarian cycle when δ-GABA_{A}R on PV+INs decrease by about 30%, γ-oscillation amplitude increases by about 25-30%. Mtor activity and hippocampal LFP, the number and duration of the various sleep episodes, and other events such as the θ-γ coupling phase amplitude coupling will be recorded during awake exploratory periods or REM sleep and will be constructed as this measure directly correlates with learning and memory performance in rodents and humans.

### Effects of Compounds on In Vitro γ-Oscillations in the CA3 Region Hippocampal Slices

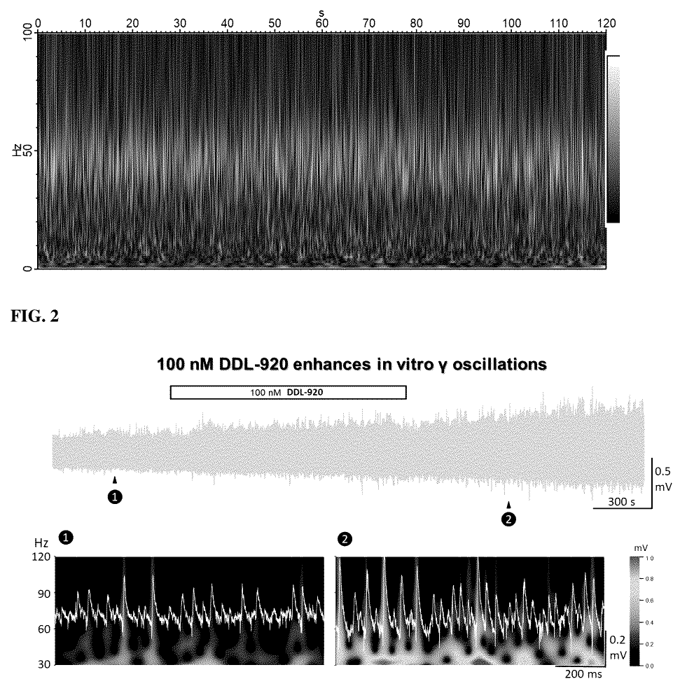

γ-oscillations were recorded in the presence of 50 nM kainic acid in horizontally cut hippocampal slices just below the CA3 pyramidal cells layer. These oscillations recorded in vitro correspond well to those recorded in vivo during spatial reference memory. In this region, the only cells with δ-GABA_{A}Rs are the PV+INs. Additional experiments were carried out in vitro to substantiate the effect of DDL-920 on γ-oscillations recorded in vitro. As shown in FIG. 2, a 10 min perfusion of 100 nM DDL-920 produced a large increase in the amplitude of γ-oscillations (upper panel, in grey). The Morlet wavelet plots (lower panels) and the raw traces (white) show the increased oscillations amplitudes in two segments of 1 s lengths taken from the recordings during the periods indicated by [symbol] and [symbol] . These findings have been repeated in a large number of experiments (FIG. 3). This figure shows the comparative effects of a 20 min perfusion of 1 or 100 nM DDL-920 or vehicle (aCSF) on the power (RMS during 60 s epochs) of in vitro γ-oscillations also indicating the number of slices. Note that there is no effect on the oscillations of vehicle perfusion, but 1 nM and 100 nM DDL-920 produce a large increase in oscillatory power.

### In Vivo PK Analysis of DDL-920 following Sub-Cutaneous (SQ) Administration

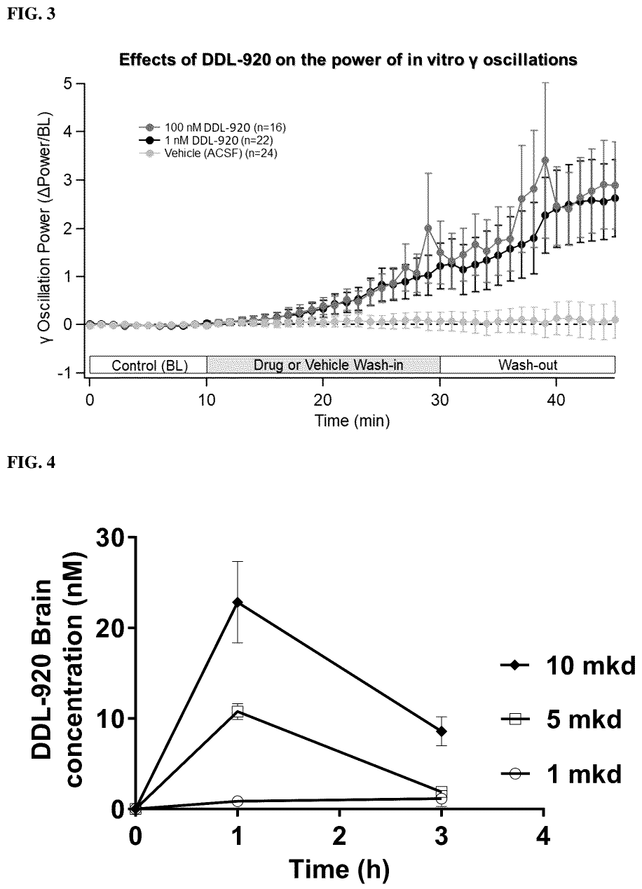

Three-months-old C57B16/J mice (n=4 for each dose) were given DDL-920 SQ at 1, 5, 10 mg/kg (mkd). Plasma and brain tissue were collected 1 and 3 hrs after. Targeted liquid LC-MS/MS assay was developed for DDL-920 using the multiple reaction monitoring (MRM) acquisition method on a 6460 triple quadrupole mass spectrometer (Agilent Technologies) coupled to a 1290 Infinity HPLC system (Agilent Technologies) with a Phenomenex analytical column (Gemini-NX 3 μm C18 110Å 100×2.0 mm). The HPLC utilized a mixture of solvent A (99.9/1 Water/Formic Acid) and solvent B (99.9/1 Acetonitrile/Formic Acid) and a gradient for the elution of the compounds (min/% B: 0/0, 5/0, 20/99, 22/99, 25/0, 35/0). FIG. 4 shows the DDL-920 concentrations achieved in the brains of mice at the indicated times after 3 different doses, 1, 5, 10 mg/kg (mkd), of SQ injections.

### Analysis of Local Field Potentials (LFP) with Emphasis on θ- and γ-Oscillations and their Coupling following SQ Administrations of 10 mg/kg DDL-920 in Mice

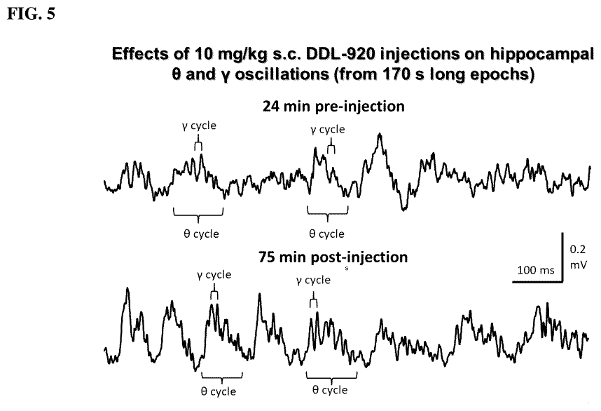

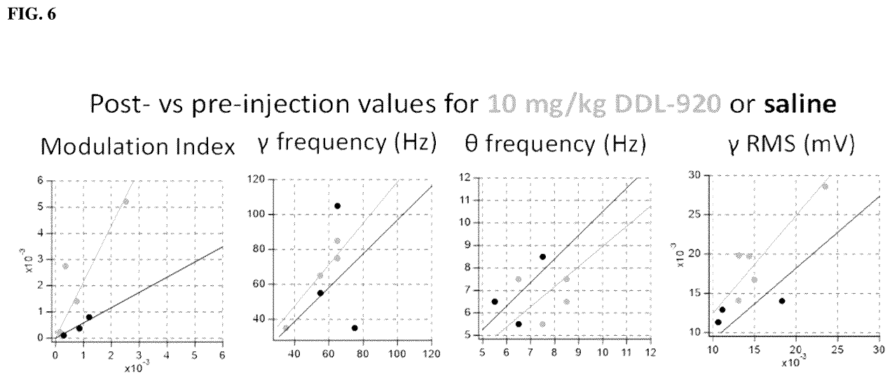

Long-term bilateral LFP recordings were performed in three-months-old C57B16/J mice with electrodes implanted in the CA1 pyramidal layer of the hippocampal formation. The LFP signals were recorded with a custom-made miniature dual headstage amplifier connected to the electrode sockets and wired to an electrical commutator (Pinnacle Technology Inc.). The signals were amplified by a 16-channel extracellular amplifier (A-M Systems; 1,000 gain), and were sampled at 2,048 s⁻¹ using a NI data acquisition board using the Igor Pro NIDaq tool (Wavemetrics, Lake Oswego, OR). The raw recordings were band pass filtered, at 5-12 Hz for θ- and 30-120 Hz for γ-oscillations. The digital FIR filtering did not change the phase of the oscillations. DDL-920 (10 mg/kg, in a volume of 0.2 ml) or vehicle (an equal volume of saline, SAL) was injected at noon, which was half-way through the 6 AM-6 PM light cycle of the animals. Measurements of the LFP signals were taken 30-60 min before the injections, and 60-120 min after the DDL-920 or SAL injections. Randomly chosen 120 s long epochs were chosen for the analysis where it was determined that e-oscillations were present. FIG. 5 illustrates a randomly chosen 1 s long epoch from the analyzed periods before and after DDL-920 injection. A couple of θ- and γ-oscillation periods are indicated by braces. Larger amplitudes of the γ-oscillations follwed the injection. Similar analyses were performed using 4 DDL-920 injected mice and 3 SAL injected mice. The four graphs in FIG. 6 indicate the modulation index (MI), γ-oscillation frequency, θ-oscillation frequency, and γ-oscillation RMS (power), respectively, plotted as the values after the injections, vs the values before the injections (DDL-920: grey n=4 mice, 5 injections; SAL: black, n=3 mice). The MI shows by how much γ amplitude for each θ frequency bin deviates from a uniform distribution, and it directly correlates with learning and memory performance in rodents and humans. The slopes of the MI and γ-oscillation power (γ RMS) are significantly (p<0.05) larger after DDL-920 injection than after SAL, indicating that these two measures have been significantly increased by the drug. It is important to note that the frequencies of the θ- and γ-oscillations remained the same following DDL-920 injection, which implies that the drug did not create new frequency domains for these oscillations.

### INCORPORATION BY REFERENCE

All publications and patents mentioned herein are hereby incorporated by reference in their entirety as if each individual publication or patent was specifically and individually indicated to be incorporated by reference. In case of conflict, the present application, including any definitions herein, will control.

### EQUIVALENTS

While specific embodiments of the subject invention have been discussed, the above specification is illustrative and not restrictive. Many variations of the invention will become apparent to those skilled in the art upon review of this specification and the claims below. The full scope of the invention should be determined by reference to the claims, along with their full scope of equivalents, and the specification, along with such variations.

We claim:

1. A compound having a structure represented by Formula I: 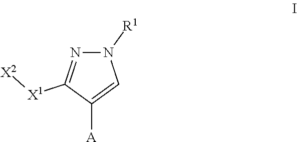
or a pharmaceutically acceptable salt thereof, wherein:
A is heterocyclyl;
X¹ is alkylene, carbonyl, N(R²), or O;
X² is aryl or heteroaryl;
R¹ is alkyl, hydroxyl, or alkoxy; and
R² is H, alkyl, or aralkyl.

2. The compound of claim 1, wherein the compound is not

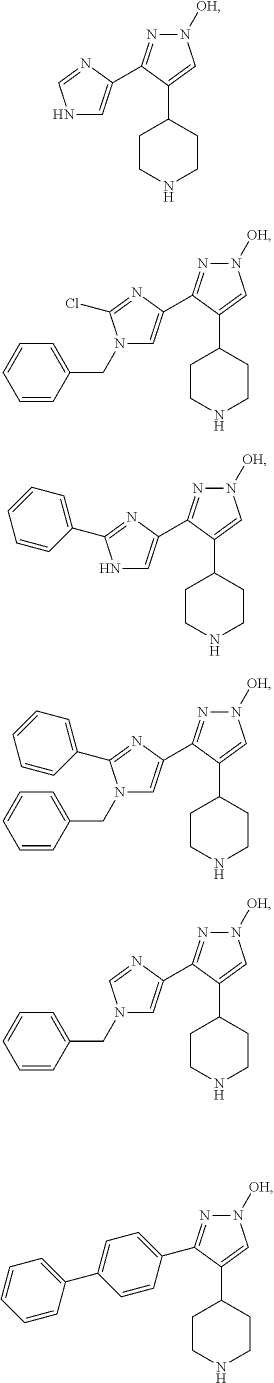 [SMILES: On1cc(C2CCNCC2)c(-c2c[nH]cn2)n1 ; On1cc(C2CCNCC2)c(-c2cn(Cc3ccccc3)c(Cl)n2)n1 ; On1cc(C2CCNCC2)c(-c2c[nH]c(-c3ccccc3)n2)n1 ; On1cc(C2CCNCC2)c(-c2cn(Cc3ccccc3)c(-c3ccccc3)n2)n1 ; On1cc(C2CCNCC2)c(-c2cn(Cc3ccccc3)cn2)n1 ; On1cc(C2CCNCC2)c(-c2ccc(-c3ccccc3)cc2)n1]

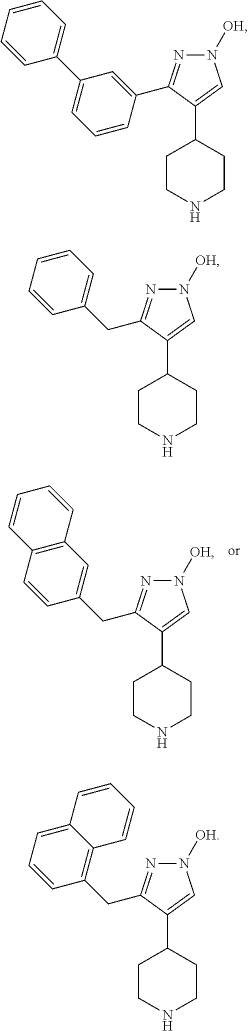 [SMILES: On1cc(C2CCNCC2)c(-c2cccc(-c3ccccc3)c2)n1 ; On1cc(C2CCNCC2)c(Cc2ccccc2)n1 ; On1cc(C2CCNCC2)c(Cc2ccc3ccccc3c2)n1 ; "or" ; On1cc(C2CCNCC2)c(Cc2cccc3ccccc23)n1]

3. The compound of claim 2, wherein the compound is not a salt of

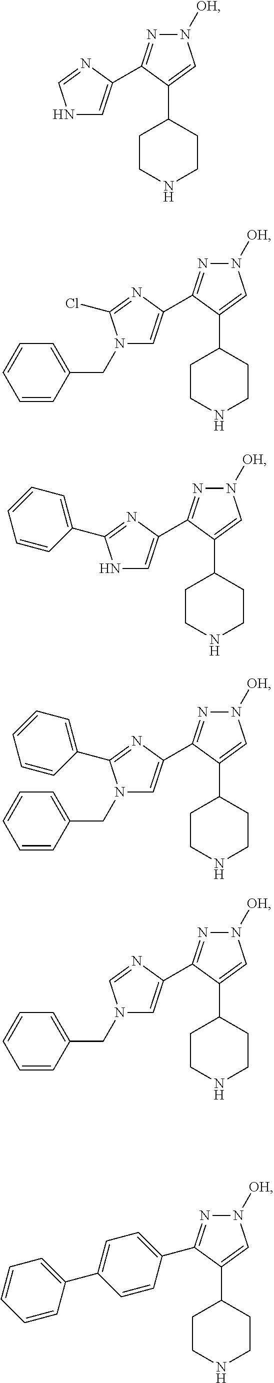 [SMILES: On1cc(C2CCNCC2)c(-c2c[nH]cn2)n1 ; On1cc(C2CCNCC2)c(-c2cn(Cc3ccccc3)c(Cl)n2)n1 ; On1cc(C2CCNCC2)c(-c2c[nH]c(-c3ccccc3)n2)n1 ; On1cc(C2CCNCC2)c(-c2cn(Cc3ccccc3)c(-c3ccccc3)n2)n1 ; On1cc(C2CCNCC2)c(-c2cn(Cc3ccccc3)cn2)n1 ; On1cc(C2CCNCC2)c(-c2ccc(-c3ccccc3)cc2)n1]

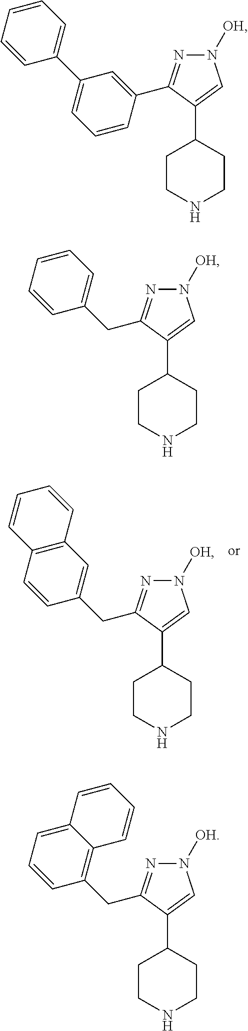 [SMILES: On1cc(C2CCNCC2)c(-c2cccc(-c3ccccc3)c2)n1 ; On1cc(C2CCNCC2)c(Cc2ccccc2)n1 ; On1cc(C2CCNCC2)c(Cc2ccc3ccccc3c2)n1 ; "or" ; On1cc(C2CCNCC2)c(Cc2cccc3ccccc23)n1]

4. The compound of any one of claims 1-3, wherein A is a 4-8 membered nitrogen containing heterocyclyl.

5. The compound of any one of claims 1-4, wherein A is azetedinyl, pyrrolidinyl, piperidinyl (e.g., N-methylpiperidinyl, N-ethylpiperidinyl, or N-propylpiperidinyl), azepanyl, or azocanyl.

6. The compound of any one of claims 1-5, wherein R¹ is hydroxyl.

7. The compound of any one of claims 1-6, wherein X¹ is alkylene (e.g., methylenyl).

8. The compound of claim 7, wherein X¹ is substituted with alkyl, alkenyl, alkynyl, halo, hydroxyl, carboxyl, acyl, acetyl, ester, thioester, alkoxy, phosphoryl, amino, amide, cyano, nitro, azido, alkylthio, cycloalkyl, alkylsulfonyl, and sulfonamide.

9. The compound of claim 7, wherein X¹ is substituted with alkyl.

10. The compound of any one of claims 1-6, wherein X¹ is carbonyl.

11. The compound of any one of claims 1-6, wherein X¹ is N(R²).

12. The compound of claim 11, wherein R² is H.

13. The compound of any one of claims 1-8, wherein the compound has a structure represented by formula Ia: 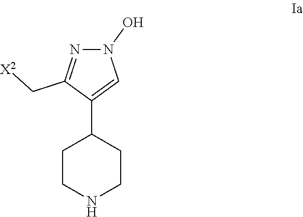
or a pharmaceutically acceptable salt thereof.

14. The compound of any one of claims 1-13, wherein X² is aryl (e.g., phenyl, naphthyl, dihydrobenzodioxinyl, or benzodioxolyl).

15. The compound of any one of claims 1-13, wherein X² is heteroaryl (e.g., quinolinyl or isoquinolinonyl).

16. The compound of any one of claims 1-15, wherein X² is substituted with alkyl, alkenyl, alkynyl, halo, hydroxyl, thiol, carboxyl, acyl, acetyl, ester, thioester, alkoxy, phosphoryl, amino, amide, cyano, nitro, azido, alkylthio, cycloalkyl, alkylsulfonyl, and sulfonamide.

17. The compound of any one of claims 1-15, wherein X² is substituted with alkyl (e.g., methyl, ethyl, propyl, isopropyl, or butyl), halo (e.g., fluoro or chloro), alkenyl (e.g., allyl), alkyloxy (e.g., methoxy), alkylthio (e.g., methylthio), thiol, or aryl (e.g., phenyl, such as methylphenyl or fluorophenyl).

18. The compound of claim 16 or 17, wherein the substituent on X² is in the para position as compared to X¹.

19. The compound of claim 1. wherein the compound is selected from 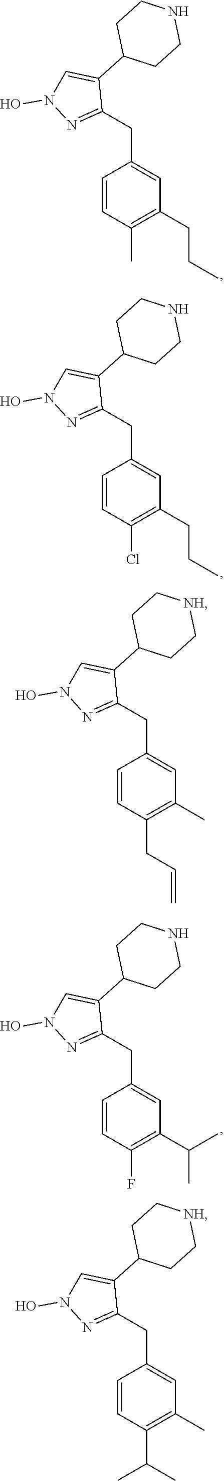 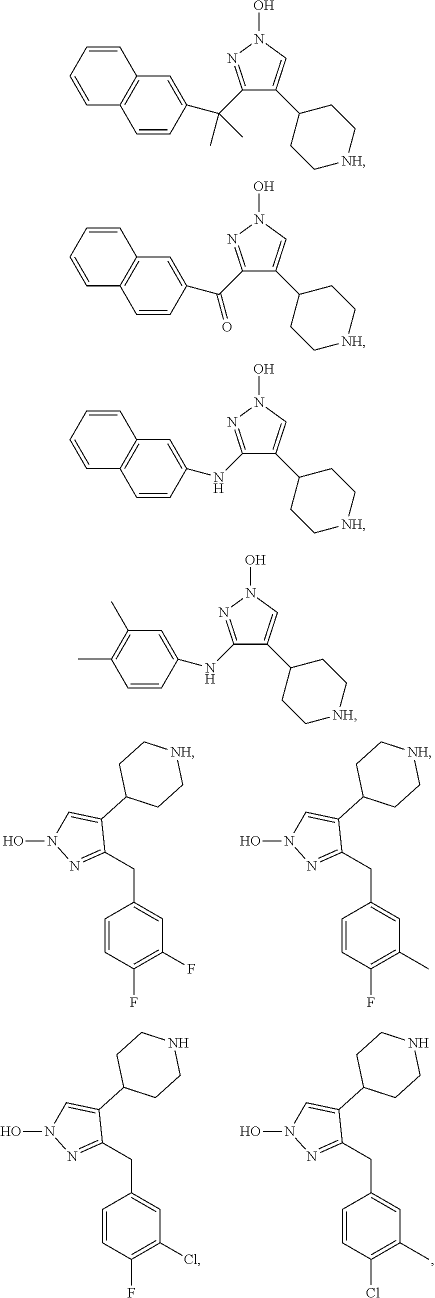 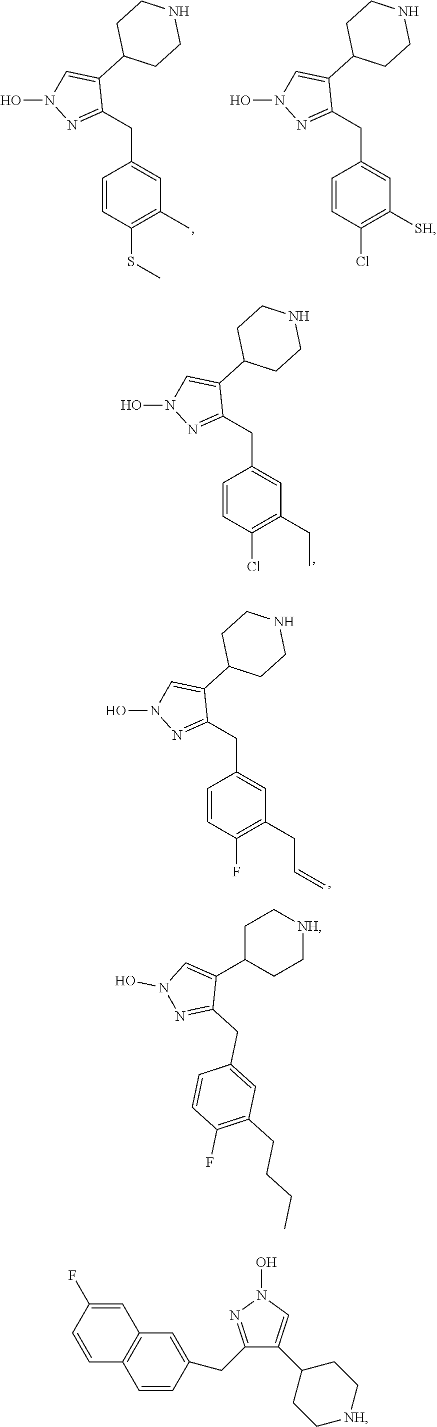 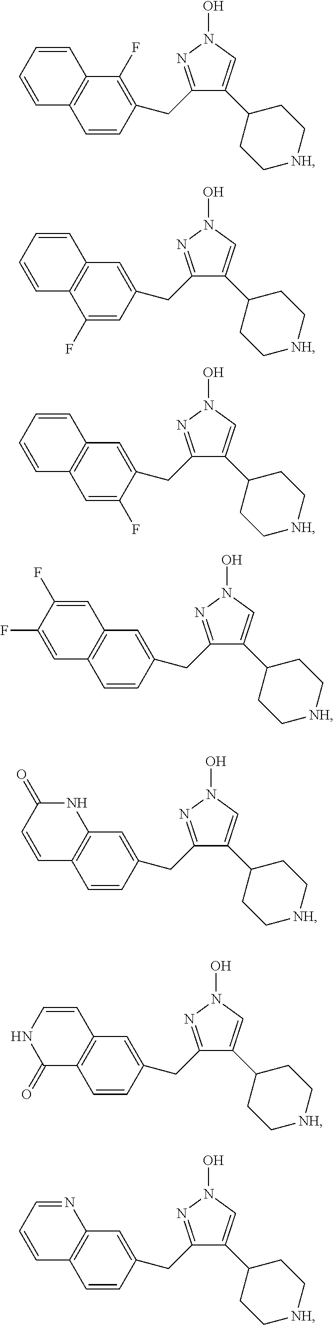 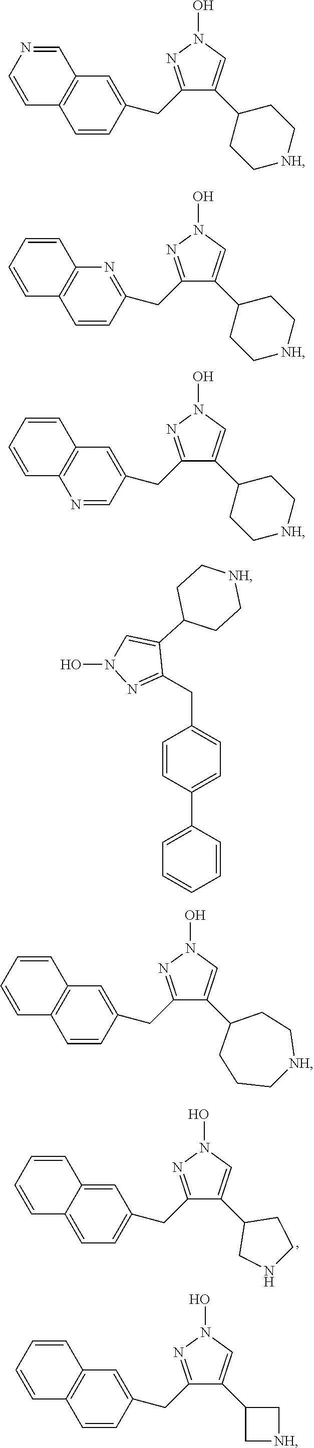 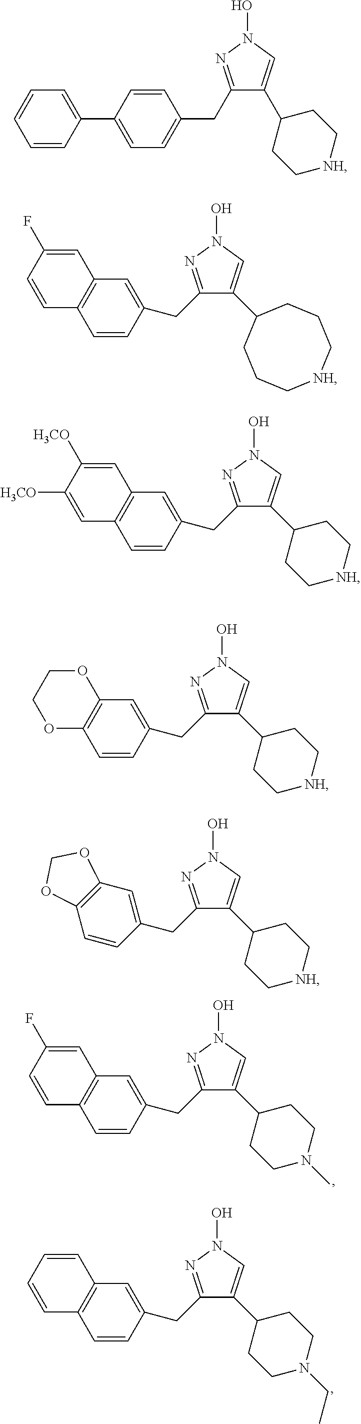 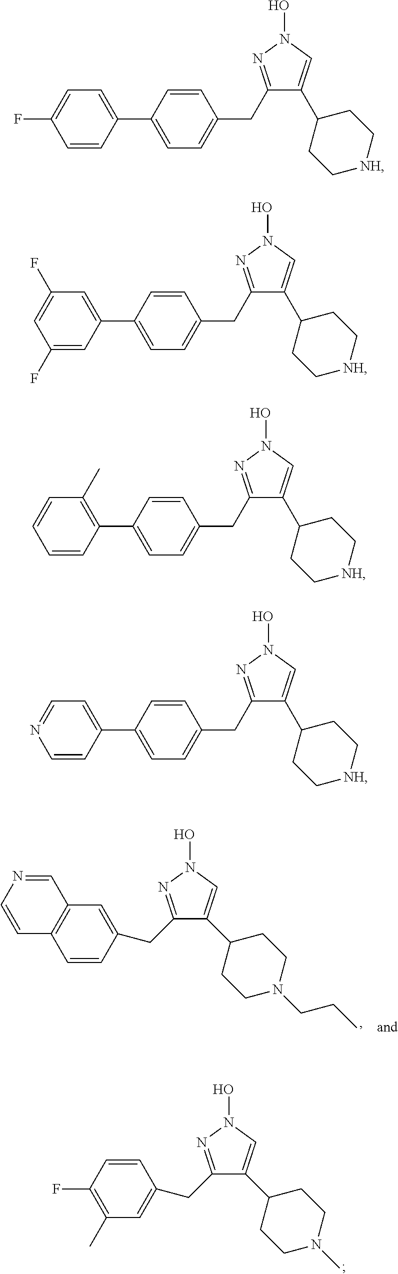
or a pharmaceutically acceptable salt thereof.

20. A pharmaceutical composition comprising a compound of any one of claims 1-19 or a pharmaceutically acceptable salt thereof and a pharmaceutically acceptable excipient.

21. A method of treating a neurodegenerative disease or disorder in a subject, comprising administering a compound of any one of claims 1-19 or a pharmaceutically acceptable salt thereof to the subject.

22. A method of treating a neurodegenerative disease or disorder in a subject, comprising administering a compound or a pharmaceutically acceptable salt thereof to the subject, wherein the compound is selected from

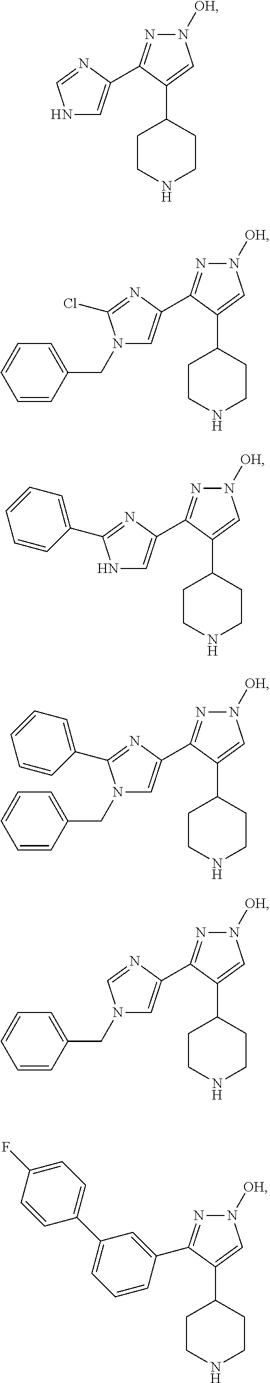 [SMILES: On1cc(C2CCNCC2)c(-c2c[nH]cn2)n1 ; On1cc(C2CCNCC2)c(-c2cn(Cc3ccccc3)c(Cl)n2)n1 ; On1cc(C2CCNCC2)c(-c2c[nH]c(-c3ccccc3)n2)n1 ; On1cc(C2CCNCC2)c(-c2cn(Cc3ccccc3)c(-c3ccccc3)n2)n1 ; On1cc(C2CCNCC2)c(-c2cn(Cc3ccccc3)cn2)n1 ; On1cc(C2CCNCC2)c(-c2cccc(-c3ccc(F)cc3)c2)n1]

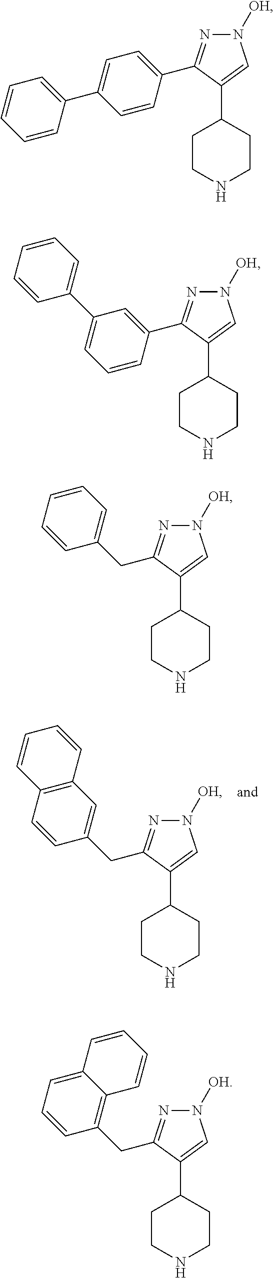 [SMILES: On1cc(C2CCNCC2)c(-c2ccc(-c3ccccc3)cc2)n1 ; On1cc(C2CCNCC2)c(-c2cccc(-c3ccccc3)c2)n1 ; On1cc(C2CCNCC2)c(Cc2ccccc2)n1 ; On1cc(C2CCNCC2)c(Cc2ccc3ccccc3c2)n1 ; "and" ; On1cc(C2CCNCC2)c(Cc2cccc3ccccc23)n1]

23. The method of claim 21 or 22, wherein the neurodegenerative disease or disorder is Alzheimer's disease, Parkinson's disease, multiple sclerosis, stroke, amyotrophic lateral sclerosis, cerebellar ataxia, frontotemporal dementia, prion disease, Huntington's Disease, cerebral ischaemia, idiopathic Morbus Parkinson, Parkinson syndrome, Morbus Alzheimers, cerebral dementia syndrome, infection-induced neurodegeneration disorders, AIDS-encephalopathy, Creutzfeld-Jakob disease, encephalopathies induced by rubiola and herpes viruses and borrelioses, metabolic-toxic neurodegenerative disorders, hepatic-, alcoholic-, hypoxic-, hypo- or hyperglycemically-induced encephalopathies, encephalopathies induced by solvents or pharmaceuticals, degenerative retina disorders, trauma-induced brain damage, cerebral hyperexcitability symptoms, cerebral hyperexcitability states, neurodegenerative syndromes of the peripheral nervous system, peripheral nerve injury, or spinal cord injury.

24. The method of claim 21 or 22, wherein the neurodegenerative disease or disorder is Autism Spectrum Disorder (ASD), Rett syndrome, intellectual disability arising from Fragile X syndrome, intellectual disability arising from variants of Fragile X syndrome, schizophrenia, depression, major depressive disorder, or post-traumatic stress disorder (PTSD).

25. The method of claim 21 or 22, wherein the neurodegenerative disease or disorder is Alzheimer's disease, Parkinson's disease, Huntington's disease, Lewy body dementia, frontotemporal dementia, amyotrophic lateral sclerosis, multiple sclerosis, progressive supranuclear palsy, or age related cognitive decline.

26. The method of claim 21 or 22, wherein the neurodegenerative disease or disorder is age related mild cognitive impairment (MCI).

27. The method of claim 21 or 22, wherein the neurodegenerative disease or disorder is Alzheimer's disease.

28. A method of increasing γ-oscillations in a subject, comprising administering a compound of any one of claims 1-19 or a pharmaceutically acceptable salt thereof to the subject.

29. A method of increasing γ-oscillations in a subject, comprising administering a compound or a pharmaceutically acceptable salt thereof to the subject, wherein the compound is selected from 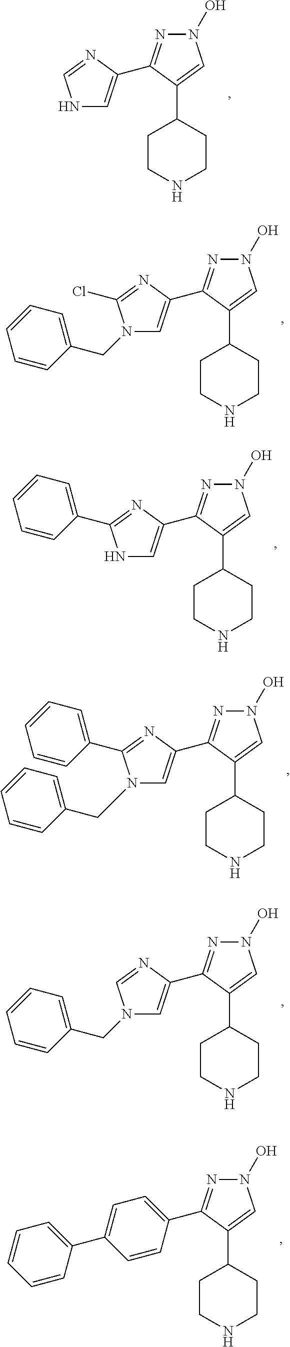 [SMILES: On1cc(C2CCNCC2)c(-c2c[nH]cn2)n1 ; On1cc(C2CCNCC2)c(-c2cn(Cc3ccccc3)c(Cl)n2)n1 ; On1cc(C2CCNCC2)c(-c2c[nH]c(-c3ccccc3)n2)n1 ; On1cc(C2CCNCC2)c(-c2cn(Cc3ccccc3)c(-c3ccccc3)n2)n1 ; On1cc(C2CCNCC2)c(-c2cn(Cc3ccccc3)cn2)n1 ; On1cc(C2CCNCC2)c(-c2ccc(-c3ccccc3)cc2)n1] [chemical structure]
